(19) United States
(12) Patent Application Publication
(10) Pub. No.: US 2022/0000767 A1
(43) Pub. Date: Jan. 6, 2022

(54) NASAL HYGIENE COMPOSITIONS, ANTIMICROBIAL TREATMENTS, DEVICES, AND ARTICLES FOR DELIVERY OF SAME TO THE NOSE, TRACHEA AND MAIN BRONCHI

(71) Applicant: SENSORY CLOUD, INC., Boston, MA (US)

(72) Inventors: David A. Edwards, Cambridge, MA (US); Thomas E. Devlin, Somerville, MA (US); Dennis Arthur Ausiello, Wellesley Hills, MA (US)

(21) Appl. No.: 17/139,401

(22) Filed: Dec. 31, 2020

Related U.S. Application Data
(60) Provisional application No. 63/048,421, filed on Jul. 6, 2020.
(60) Provisional application No. 63/121,448, filed on Dec. 4, 2020.
(60) Provisional application No. 63/130,099, filed on Dec. 23, 2020.

(51) Int. Cl. A61K 9/00; A61K 9/12; A61K 47/02

(52) U.S. Cl. CPC A61K 9/0043; A61K 47/02; A61K 9/0078; A61K 9/12

(57) ABSTRACT

Salt-based non-therapeutic hygienic formulations or compositions or therapeutic formulations or compositions, for example those rich in calcium, are effective against airborne pathogens, suppressing virus shedding, for instance by increasing a surface viscoealasticity of airway lining fluid. Associated apparatus, methods and articles are used to deliver salt-based non-therapeutic hygienic formulations or compositions as hygienic treatments to the upper respiratory tract. Associated apparatus, methods and articles are used to deliver salt-based therapeutic antimicrobial formulations or compositions to the upper respiratory tract. For example, nasal delivery of calcium-rich salines with aerosol droplet size of around 10 μm (e.g., 7 μm-15 μm, or more preferably 9 μm-10 μm) may advantageously limit distribution to the nose and upper airways of the respiratory tract, suppressing bioaerosol generation. A nebulizer may deliver the aerosol into free space, or into a partially enclosed volume, and the composition naturally inspired by one or more subjects.

### FIELD

This disclosure generally relates to suppression of exhaled aerosol particles from the upper airways of the human respiratory tract via nasally administered salt-based formulations or compositions, for example calcium rich salt-based formulation or compositions with a mass median diameter of around 10 microns, and treatment protocols, devices, and articles suitable for the delivery of salt-based compositions as aerosols to the nose, trachea and main bronchi of a respiratory tract of a subject.

### BACKGROUND

### Description of the Related Art

Various illnesses are caused by viruses, bacteria and other inhaled foreign particles. At least some viruses, for example various variations of the flu viruses and variations of corona viruses are communicated, at least in part, via respiratory transmission. Examples of airborne bacterial infections include tuberculosis. In one mechanism, an individual inflected with the microbe may shed the microbe through breakup of airway lining fluid during respiration, coughing, sneezing, talking or even singing, subjecting others to the airborne microbe. The shed microbe may also be drawn inwardly, deeper into the respiratory tract, of the subject, for example into the lungs. Inhaled particles from the environment, for instance soot particles, may land on the upper airway lining mucus. Breakup of the mucus into respiratory droplets can carry these particles deeper into the lungs, promoting allergic responses or other respiratory ailments.

Severe acute respiratory syndrome-coronavirus-2 (SARS-CoV-2) transmits through the air by a combination of the large droplets exhaled when people cough or sneeze, and by the very small droplets people generate in their airways when they naturally breathe. How exhaled respiratory droplets vary between individuals, evolves over time within individuals, and changes with the onset and progression of COVID-19 infection is critical to clarifying the nature of COVID-19 transmission—and other highly-communicable airborne respiratory diseases, such as influenza and tuberculosis.

The delivery of therapeutic substances to the nasal epithelium and upper airways to modulate human health, as in the delivery of active substances for relieving congestion, or symptoms related to asthma, generally involves the active (forced) delivery of dry or liquid formulations to the nose via a spray, metered dose inhaler, or dry powder inhaler. The use of these substances to fight against infectious diseases is generally limited to antibiotic or anti-viral drugs. The delivery of purely nasal hygiene substances, as in sodium chloride, for cleaning mucus is also common. Applicant is unaware of any nasal hygiene specifically targeted against infectious diseases, in the way that the washing of hands or the wearing of masks provides protection against infection.

The ability to return to normal activities in the face of an airborne infectious disease pandemic of the kind posed by COVID-19 may hinge on the ability to provide hygienic protection against airborne infection in the vicinity of the nose, trachea and main bronchi where infection often begins and where considerable bioaerosol shedding occurs. Such may be important on an individual basis, as well as in crowds, for instance where social distancing cannot be ensured. This same hygienic protection might be beneficial for other airborne infectious diseases, such as influenza, and also useful to promoting respiratory health for individuals exposed to high levels of air pollution.

### BRIEF SUMMARY

Given the potential for widespread illness via the inhalation of foreign particles, as evidenced by the ongoing COVID-19 pandemic, new approaches that effectively deliver hygienic and therapeutic substances in order to reduce the generation of respiratory droplets in the upper airways, and therefore the suppression of exhaled droplets or bioaerosols, are desirable.

Generation of respiratory droplets in exhaled breath can occur by the force of the fast air flows that occur in the upper airways when we breathe, talk, cough and sneeze. At peak inspiratory flows during normal breathing, air speeds in the trachea and main bronchi can reach turbulent velocities. The rush of air over the thin (5 μm to 10 μm) mucus layer lining the upper airways can break up the mucus surface into small droplets in the way strong winds produce breakup and spray on the surface of the ocean. The nature and extent of this droplet breakup is dependent on the surface properties of the mucus itself. Among properties most influencing droplet generation and droplet size are surface viscoelasticity (which resists the stretching of mucus surface on breakup) and surface tension (which lowers the energy expended in small droplet creation). In airway lining mucus, both properties vary with lung surfactant type and concentration, surface particulate concentration, as well as with composition and structure of mucus in close proximity to air surfaces. Particulate accumulation, e.g., by breathing polluted air or pathogen proliferation, or surfactant and mucin compositional and structural changes, driven in part by physiological alterations of the human condition—including diet, aging, and COVID19 infection itself—may therefore be anticipated to alter droplet generation and droplet size during acts of breathing.

The scientific response to the COVID-19 pandemic has largely focused on the development of curative drugs and preventive vaccines. In the wait for a cure or collective immunity, it may be advisable for the scientific community to additionally focus on management of COVID-19; in part through the diminution of respiratory droplet generation as reflected in the numbers of exhaled breath particles. Notably, typical masks do not stop submicron particles, which are the most numerous of all in air expelled by an infected individual. For example, it is believed that approximately 85% of (inhaled and exhaled) respiratory particles are in the submicron size range. Beyond more effective masks and more sophisticated social distancing rules, approaches to stabilize airway lining mucus and retain mucus clearance function might be particularly useful.

Notably, exhaled aerosol numbers appear to be not only an indicator of disease progression, but a marker of disease risk in non-infected individuals. Monitoring as a diagnostic might also be an important strategy to consider in the control of transmission and infection of COVID-19 and other respiratory infectious diseases, including influenza.

The nasal administration of physiological salts appears particularly effective at reducing airborne particles from exhaled breath including the sub-micron aerosolized particles that are ineffectively filtered by cloth face masks. For example, nasal application of a drug-free calcium-enriched nasal salt interacts with airway lining mucus to cleanse the airways of bioaerosols which may reduce exhaled aerosol particles up to 99%, with an overall reduction of exhaled particles in a large cohort of human subjects of around 75%. The cleansing may not only reduce the exhalation of particles, but may also reduce the inhalation of particles further into the airways (e.g., lower respiratory tract). The nasal administration of physiological salts can be an important addition to current COVID-19 hygiene protocols of mask wearing, hand washing, and social distancing. The nasal administration of physiological salts adds to the efficacy of masks at reducing the penetration of respiratory droplets into the lungs or back into the environment; and provides an added layer of defense for when mask wearing is not possible.

The global crisis caused by the rapid and tenacious spread of COVID-19 points to the inadequacy of current defenses against airborne infectious diseases. Nasal administration of physiological salts, and in particular, calcium-enriched physiological salts may be employed to address the challenge that the air route of infection and transmission presents to public health. There is a particular need for purely hygienic compositions and methods for cleaning the airways of droplets of airway lining fluid, as are generated during natural breathing, talking, coughing and sneezing. In a study of 10 human volunteers (5 younger than 65, 5 older than 65), the 5 to 15 second delivery of a nasal saline comprising calcium and sodium salts with a 9-10 μm mean diameter quickly (e.g., within 15 minutes) and durably (e.g., up to at least 6 hours) diminishes exhaled particles from the human airways without appreciably penetrating the lungs beyond the trachea and main bronchi. Being predominantly smaller than 1 μm, these exhaled particles are largely below the size effectively filtered by conventional masks. By contrast, in the same study, the delivery of a similar calcium-enriched nasal saline with 2-4 μm mean diameter droplets required several minutes to administer and led to less uniform suppression of exhaled particles from the human airways while also penetrating the lungs beyond the trachea and main bronchi. The delivery of isotonic normal saline (sodium chloride) by small aerosol (2-4 μm droplets) to the lungs has a more modest effect on suppression, as shown in the same study as well as in a 2004 study. Delivering isotonic normal saline to the nose by large droplets ranging between approximately 10 μm and 300 μm, with a mean diameter of around 40 to 50 μm, in the same study of 10 human volunteers, led to no effect on exhaled aerosol. However, in a recent study, delivery of this same aerosol with similar large droplet diameter, tilting of the head to permit post-nasal drip showed significant suppression of exhaled aerosol. These results are consistent with the known deposition patterns of inhaled aerosols. Particularly, droplets in the range of 1 to 5 μm tend to penetrate and deposit throughout the respiratory tract including in the alveolar region of the lungs. The generation of a given mass for deep lung delivery with small droplets in this size range requires more energy than the generation of the same mass for delivery within droplets of say 10 μm in size, and therefore requires, for a given energy input, more time. Inhaled droplets of around 10 μm in diameter predominantly deposit in the nasal pharynx and in the trachea and main bronchi. On the other hand droplets of 15 to 300 μm can be sprayed into the nose but land in the nose and do not penetrate the trachea, while it is known that by post-nasal drip solution deposited in the nose can drip into the trachea and main bronchi. While other divalent cations can be delivered to the airways with an aim to suppress respiratory droplet formation, calcium is particularly desirable in that it is present in the body at a relatively high concentration. Magnesium is also in the body while at lower concentrations (roughly 5 to 10 times lower than calcium concentrations) and also acts on negatively charged molecules such as mucins and alginates more slowly than calcium. Other divalent and multivalent cations may be effective but are either foreign to the body, as in the case of chitosan, or present in the body, as in the case of iron, while introducing other complex biochemical consequences.

The suppression of exhaled droplets by the nasal delivery of calcium-rich salines—with and without sodium chloride, and other additives including lavender, cinnamon, lemongrass, and alcohol—with aerosol droplet diameter of around 10 μm (e.g., 7 μm-15 μm) suggests the upper airways (i.e., the nose, trachea and main bronchi) as a primary source of bioaerosol generation. The suppression effect is especially pronounced (99%) among those who exhale large numbers of particles. High particle exhalation appears to correlate with advanced age and body mass index (BMI) as well as with lung infection and prolonged exposure to high fine particle aerosol burden in the atmosphere.

A new hygienic practice of “airway hygiene” using a calcium-rich saline nasally-administered solution is proposed, to complement the widely-recommended washing of hands with ordinary soap, use of a face mask, and social distancing. Airway hygiene might be immediately introduced next to these other hygienic measures.

The combination of hygiene and therapy, whether through combinations of salts that have been shown to produce antimicrobial effects or through other therapeutically active substances, is also desirable.

There is also a need for therapeutic compositions and methods for treating the airways of individuals who are known or suspected of having an airborne illness (e.g., infection of COVID-19 and other respiratory infectious diseases, including influenza), or who have been exposed to other individuals who are known or suspected of having an airborne illness.

A new therapeutic practice of nasal application of a calcium-rich saline aerosol, with or without other salts that have been shown to produce antimicrobial effects or through other therapeutically active substances, to the upper respiratory tract is described. Nasal application of physiological salts, and in particular application of an aerosol of droplets containing calcium rich salts (e.g., calcium chloride) in sizes that constrain the aerosol predominately in the upper respiratory tract may advantageously produce antimicrobial and/or anti-pathogen effects.

In particular, a mister or nebulizer that delivers to the upper airway site of respiratory droplet formation an aerosol that has a high concentration of calcium may be particularly effective. The ions of the calcium rich salt may associate with mucins on the surface of the airway lining mucus, strengthening resistance to the breakup of mucus. This may advantageously clean the airways of the respiratory droplets that can carry infection and insoluble environmental contaminants.

Otherwise a calcium rich salt solution applied to the nose by a spray or an installation, combined with a leaning back of the head or reclined position that promotes post-nasal drip, is another method to deliver to the upper airway site of respiratory droplet formation a high concentration of calcium or other multi-valent cationic molecule.

Hygienically and therapeutically active substances deposit in the nose, depending on the nature of the delivery system and technique, with some associated degree of efficiency. This efficiency can be measured as a fraction of “delivered dose” to “nominal dose.” Delivery of substances to the nasal epithelium occurs in two ways. The first, ortho-nasal scent delivery, occurs by sniffing substances in the atmosphere, e.g., directly via the nostrils or nasal vestibule. The second, retro-nasal scent delivery, occurs by the natural diffusion and convection of substances in the mouth into the nasal passages via the oropharynx. This latter delivery is referred to as retro-nasal olfaction, and is promoted by exhalation.

Described herein are new salt-based hygienic and/or antimicrobial formulations or compositions that are effective against airborne pathogens and other airborne contaminants, and associated apparatus, methods and articles for delivery of salt-based antimicrobial and/or anti-contagion formulations or compositions. The described salt-based hygienic and/or antimicrobial formulations or compositions, apparatus, methods and articles can advantageously be employed to suppress or otherwise reduce the shedding of aerosol particles of airway lining fluid (bioaerosol), either on an individual basis, or in groups or crowds of individuals. The described approaches employ a combination of ortho-nasal and retro-nasal delivery, the former occurring on inspiration of the physiological salt solutions, and the latter on exhalation of these same salt solutions. The salt-based formulations or compositions are formulated in readily-soluble solutions applied to the nose as an installation or a spray, or in the form of aerosolized water droplets that have a mass median droplet diameter range of approximately 7 microns to approximately 15 microns, with a standard deviation of less than 5 microns; alternatively the mass median droplet diameter is approximately 9 to approximately 10 microns, approximately 9.5 microns, or approximately 10 microns, with a standard deviation of less than 1 micron. These droplet diameter ranges are advantageously too large for significant penetration into the lungs, while small enough to be carried into the trachea and main bronchi of the respiratory tract via the nose.

The salt-based hygienic and/or antimicrobial formulations or compositions may preferably be rich in calcium or magnesium (e.g., calcium or magnesium chloride). Suitable salt-based hygienic and/or antimicrobial formulations or compositions rich in calcium or magnesium may, for example, include: salt solutions containing 1%, 2%, 3%, 4%, 5%, 6%, 7% or 8% CaCl₂) or MgCl₂; alternatively about 1 to about 10%, about 4% to about 10%, 1.0-8.0%, 1.0-6.0%, 1.0-2.0%, or 4.0-6.0% CaCl₂) or MgCl₂. These CaCl₂) or MgCl₂ solutions might additionally contain NaCl, and may, for example, include: solutions containing 0.1%, 0.5%, 1.0%, or 1.5% NaCl; alternatively 0.1-1.5%, 0.5-1.5%, or 0.1-0.5% NaCl. In another embodiment, the salt-based compositions do not have any NaCl in the compositions. The percentages may be wt % based on the total amount of the salt-based composition. As the examples demonstrate, the salt-based composition may comprise 4.72% CaCl₂ and 0.31% NaCl, or 1.29% CaCl₂) and 0.9% NaCl.

The salt-based solution may also contain one or more preservatives. The preservatives may include any preservative known in the art that would not otherwise interfere with the chemistry of the salts in the salt-based formulation. As one skilled in the art would appreciate, preservatives are on the FDA list of non-active agents. Suitable preservatives include benzalkonium chloride, benzyl alcohol, and benzoic acid. The preservative can be added in amounts known to those of skill in the art, for instance, 0.05-0.2 wt %, or about 0.1 wt %. As an alternative to the addition of preservatives, the salt-based solution might also have low pH, through the addition of HCl or by some other means, e.g., pH in the range of about 2 to about 6; alternatively, about 2 to about 5; about 2 to about 3; or about 2.5. The HCl acid may be added with a citric acid buffer.

Alternatively, compositions of CaCl₂) with or without NaCl containing lavender, cinnamon, lemongrass, and ethanol, among other essential oils and flavor extracts, are all effective. Essential oil, fragrance oil, and flavor extract compositions can take any of a large variety of forms, and may be mixed with water (e.g., distilled or sterilized water). For example, begin with a vial of water 25 milliliters. Add 0.025 to 1 milliliter of essential oil, fragrance oil or flavor extract, as in cacao oil, caramel oil, cinnamon bark oil, coffee oil, eucalyptus oil, palm oil, fig oil, grapefruit oil, hazelnut oil, honeydew melon oil, lavender or spike lavender oil, lemongrass oil, lime oil, black or green pepper oil, peppermint oil, rosemary oil, strawberry oil, smoke oil, tobacco vanilla oil, vanilla oil, chocolate extract, anise extract, and/or linalool. As an example, where the mass of cloud dispersed before the nose is less than 100 milligram total mass, the total quantity of ginger may be less than approximately 100 micrograms.

Application may be simple, for example one or more deep nasal inspirations may diminish exhaled aerosol by up to 75% or even 99% for up to six hours after administration. A protocol for administration may, for example include application on arrival at a location (e.g. worksite) prior to masking up. An initial application (e.g., two deep nasal inspirations) may be administered by a staff member or other personal assigned to the specific task. A second application (e.g., two deep nasal inspirations) may follow, for example during a meal (e.g. lunch) break. The second application may, for example, be self-administered. Self-administration may be performed from freely-accessible wall and/or table mounted nebulizers or misters, which may advantageously include hand sanitizer dispensers attached or positioned proximate to the nebulizers or misters. Alternatively, each individual (e.g., employee) may be supplied with a personal nebulizer or mister and an adequate supply of calcium rich solution or dry powder to allow the individual to self-administer the calcium rich airway hygiene, for example three times a day. Training may advantageously be provided, particularly where self-administration is employed.

Various apparatus are also describe herein which allow the portable, discrete delivery of salt-based hygienic and/or antimicrobial formulations or compositions, enhancing efficiency of delivery to humans and other animals on an individual basis. Advantageously, the apparatus is configured to be portable, allowing the user to have the benefit of on demand delivery, in a wide variety of environments, to suppress virus shedding.

Various apparatus are also described herein which allow the mass delivery of salt-based formulations or compositions to groups (e.g., two or more individuals, crowds, lines of individuals), enhancing efficiency of delivery to humans and other animals on a group basis. Such may be fixed, or portable devices. Such may be suitable for use with crowds at stadiums, other venues, and/or at various events.

Further, there is a need for diagnostic methods and apparatus that monitor respiratory droplet shedding from individuals, for example individuals who are known or suspected of having an airborne illness (e.g., infection of COVID-19 and other respiratory infectious diseases, including influenza), or who have been exposed to other individuals who are known or suspected of having an airborne illness, or who breathe for long durations polluted air. A diagnostic method may include: sampling exhaled breath for a subject, determining a metric that characterizes an amount of exhaled respiratory droplets shed in the exhaled breath, and correlating the metric with a category that indicates at least one of: a level of illness risk and/or a level of transmission or transmissibility risk or a level of suggested quarantine precautions to be taken. The metric may, for example, take the form of a count or approximate count of exhaled respiratory droplets and/or pathogen presence, or another representation of an aerosol number. Sampling the exhaled breath may be performed over a defined number of breathes or over a defined period of time. Correlation may be performed with respect to a representative sampling of breath samples taken from a representative sample of a population.

Accordingly, one implementation may be summarized as a composition of aerosol droplets comprising a salt-based composition comprising (a) from about 1% to about 10% by weight calcium chloride and/or magnesium chloride in water; and (b1) a preservative selected from the group consisting of benzalkonium chloride, benzoic acid, and benzoyl alcohol, or (b2) an acid in an amount sufficient to reduce the pH of the salt-based composition to about 2 to about 6. Either the (b1) preservative or the (b2) acid may be present in this embodiment. The droplets have a mass median droplet diameter ranging from approximately 7 microns to approximately 15 microns.

Another implementation may be summarized as a composition comprising (a) a dry powder containing calcium and/or magnesium chloride; and (b) a sterile solution of a water-based composition comprising (1) a preservative selected from the group consisting of benzalkonium chloride, benzoic acid, and benzoyl alcohol, or (2) an acid in an amount sufficient to reduce the pH of the salt-based composition to about 2 to about 6. The dry powder can be mixed with the water-based composition to form a salt-based composition.

Another implementation may be summarized as a method of administering a formulation or composition to the nose, trachea, and main bronchi of a respiratory tract of a subject. The method comprises generating an aerosol of droplets in a space from which the aerosol is naturally inspirable by the subject, in the nose, trachea, and main bronchi of the respiratory tract of the subject, without any application of force. The aerosol of droplets comprises a salt-based composition comprising calcium chloride and/or magnesium chloride in water, and the droplets have a mass median droplet diameter ranging from approximate 7 microns to approximately 15 microns.

Another implementation may be summarized as a method of suppressing the exhalation of particles in an upper airway of a respiratory tract of a subject. The method comprises generating an aerosol of droplets, and administering the aerosol of droplets to the airway lining fluid in the nose, trachea, and main bronchi of the subject, thereby suppressing the exhalation of particles in the upper respiratory tract of the subject. The aerosol of droplets comprise a salt-based composition comprising calcium chloride and/or magnesium chloride in water droplets, the droplets have a mass median droplet diameter ranging from approximately 7 microns to approximately 15 microns, and the droplets are suspended in a standing cloud.

Another implementation may be summarized as a delivery system operable to delivery of a purely hygienic or antimicrobial formulation or composition to the nose, trachea and main bronchi of a respiratory tract of a subject. The delivery system comprises a reservoir having at least one wall which at least partially delimits an interior of the reservoir from an exterior thereof, the reservoir having a port that provides a fluidly communicative path between the interior of the reservoir and an exterior thereof, the reservoir which at least in use holds the hygienic or antimicrobial formulation or composition comprising a quantity of water and at least calcium chloride and/or magnesium chloride dissolved in the water. The delivery system also includes at least one nebulizer delivery device, the at least one nebulizer delivery device comprising a reservoir and an actuator, and the actuator controllably operable on the active substance media to cause formation of an aerosol comprising readily-soluble droplets that have a mass median diameter range of approximately 7 microns to approximately 15 microns and comprising at least the calcium chloride dissolved in the quantity of water.

Another implementation may be summarized as a kit to suppress the exhalation of particles in an upper airway of a respiratory tract of subjects. The kit comprises a measured quantity of calcium chloride and/or magnesium chloride; a container sized to receive a defined quantity of water to dissolve the calcium chloride therein; and instructions.

Another implementation may be summarized as a method of administering a formulation or composition to the nose, trachea, and main bronchi of a respiratory tract of a subject. The method comprises generating an aerosol of droplets in a space from which the aerosol is naturally inspirable by the subject, in the nose, trachea, and main bronchi of the respiratory tract of the subject, without any application of force. The aerosol of droplets comprises a salt-based composition comprising calcium chloride and/or magnesium chloride in water. The method of administering the formulation or composition to the nose, trachea, and main bronchi of a respiratory tract of the subject is achieved by spraying the salt-based composition in the nose of the subject while the subject has their head leaning back or is in a reclined position that promotes post-nasal drop.

Another implementation may be summarized as a composition of aerosol droplets comprising a salt-based composition, comprising (a) from about 1% to about 5% by weight calcium chloride in water; and (b1) a benzalkonium chloride preservative, or (b2) an acid in an amount sufficient to reduce the pH of the salt-based composition to about 2 to about 3. Advantageously, this method is not limited by the droplet size, although the ranges described can still be effectively used with the method, for instance in a 20-30 mg dosage range.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1A is an isometric view of a delivery device delivering an aerosol of a salt-based hygienic and/or antimicrobial formulation or composition into an unconfined space or volume to be inspired by a subject, according to at least one illustrated implementation.

FIG. 1B is an illustrative diagram of various sequential acts performed in using a delivery device to deliver an aerosol of a salt-based hygienic and/or antimicrobial formulation or into either a unconfined or free space or volume, or into a confined space or volume (e.g., mask, tumbler, glass, vial beaker or other container), to be inspired by a subject, according to at least one illustrated implementation.

FIG. 2A is a bar graph showing particles exhaled from two human volunteers prior to dosing with a salt-based hygienic and/or antimicrobial formulation or composition who exhibited relatively high virus shedding, according to at least one illustrated implementation.

FIG. 2B is a bar graph showing particles exhaled from eight human volunteers prior to dosing with a salt-based hygienic and/or antimicrobial formulation or composition who exhibited relatively average virus shedding, according to at least one illustrated implementation.

FIG. 3 is a line graph showing particles exhaled from the ten human volunteers after dosing with a salt-based hygienic and/or antimicrobial formulation or composition, according to at least one illustrated implementation.

FIG. 4A is a graph showing particles exhaled per subject following dosing with a salt-based hygienic and/or antimicrobial formulation or composition in comparison to a placebo control, according to at least one illustrated implementation.

FIG. 4B is a graph showing particles exhaled per subject following dosing with a salt-based hygienic and/or antimicrobial formulation or composition in comparison to a placebo control, according to at least one illustrated implementation.

FIG. 4C is a graph showing particles exhaled per subject following dosing with a salt-based hygienic and/or antimicrobial formulation or composition in comparison to a placebo control, according to at least one illustrated implementation.

FIG. 4D is a graph showing particles exhaled per subject following dosing with salt-based hygienic and/or antimicrobial formulation or composition in comparison to a placebo control, according to at least one illustrated implementation.

FIG. 4E is a graph showing particles exhaled per subject following dosing with a salt-based hygienic and/or antimicrobial formulation or composition in comparison to a placebo control, according to at least one illustrated implementation.

FIG. 4F is a graph showing particles exhaled per subject following dosing with a salt-based hygienic and/or antimicrobial formulation or composition in comparison to a placebo control, according to at least one illustrated implementation.

FIG. 4G is a graph showing particles exhaled per subject following dosing with a salt-based hygienic and/or antimicrobial formulation or composition in comparison to a placebo control, according to at least one illustrated implementation.

FIG. 4H is a graph showing particles exhaled per subject following dosing with a salt-based hygienic and/or antimicrobial formulation or composition in comparison to a placebo control, according to at least one illustrated implementation.

FIG. 4I is a graph showing particles exhaled per subject following dosing with salt-based hygienic and/or antimicrobial formulation or composition in comparison to a placebo control, according to at least one illustrated implementation.

FIG. 5 is schematic view of a portion of a delivery device, including a nebulizer which can include a screen and at least one of a piezo-electric element, solenoid or electric motor physically coupled to move the screen, the device also including one or more of a radio, a transducer or sensor and a switch communicatively coupled to a control system, for example a microcontroller and memory, and operably coupled to control operation of the nebulizer, according to at least one illustrated implementation.

FIG. 6A is an exploded view of a delivery device of FIG. 5 to deliver a mist, a cloud, or an aerosol comprising a salt-based hygienic and/or antimicrobial formulation or composition that is effective against airborne pathogens, according to at least one illustrated embodiment.

FIG. 6B is a perspective view of the delivery device to deliver a mist, a cloud, or an aerosol comprising a salt-based hygienic and/or antimicrobial formulation or composition that is effective against airborne pathogens of FIG. 6A, according to at least one illustrated embodiment.

FIG. 6C is a side view of the delivery device to deliver a mist, a cloud, or an aerosol comprising a salt-based hygienic and/or antimicrobial and/or formulation or composition that is effective against airborne pathogens of FIGS. 6A and 6B, according to at least one illustrated embodiment.

FIG. 6D is a cross-sectional side view of the delivery device to deliver a mist, a cloud, or an aerosol comprising a salt-based hygienic and/or antimicrobial formulation or composition that is effective against airborne pathogens of FIGS. 6A-6C, according to at least one illustrated embodiment.

FIG. 6E is a side view of components of the delivery device to deliver a mist, a cloud, or an aerosol comprising a salt-based hygienic and/or antimicrobial formulation or composition that is effective against airborne pathogens of FIGS. 6A-6D, according to at least one illustrated embodiment.

FIG. 6F is another side view of components of the delivery device to deliver a mist, a cloud, or an aerosol comprising a salt-based hygienic and/or antimicrobial formulation or composition that is effective against airborne pathogens of FIGS. 6A-6D, according to at least one illustrated embodiment.

FIG. 7A is a rear view of a printed circuit board and associated components coupled thereto for use in the delivery device to deliver a mist, a cloud, or an aerosol comprising a salt-based hygienic and/or antimicrobial formulation or composition that is effective against airborne pathogens of FIGS. 6A-6F, according to at least one illustrated embodiment.

FIG. 7B is a side view of the printed circuit board and associated components of FIG. 7A, according to at least one illustrated embodiment.

FIG. 7C is a front view of the printed circuit board and associated components of FIGS. 7A and 7B, according to at least one illustrated embodiment.

FIG. 7D is a perspective view of the printed circuit board and associated components of FIGS. 7A-7C, according to at least one illustrated embodiment.

FIG. 7E is a rear view of the printed circuit board of FIGS. 7A-7D, without the associated components coupled thereto of FIGS. 7A-7D, according to at least one illustrated embodiment.

FIG. 7F is a side view of the printed circuit board of FIGS. 7A-7D, without the associated components coupled thereto of FIGS. 7A-7D, according to at least one illustrated embodiment.

FIG. 7G is a front view of the printed circuit board of FIGS. 7A-7D, without the associated components coupled thereto of FIGS. 7A-7D, according to at least one illustrated embodiment.

FIG. 7H is a front view of an alternative configuration of the printed circuit board of FIGS. 7A-7D, without the associated components coupled thereto of FIGS. 7A-7D, according to at least one illustrated embodiment.

FIG. 8 illustrates the general design of the protocol for the three study sites, specifically the GRCC study site, discussed in the experimental section.

FIGS. 9A, 9B and 9C show the results, in terms of exhaled aerosol particle numbers and sizes, for the 40 human subject volunteers in Bangalore (Figure (A), 120 human volunteers in Grand Rapids (FIG. 9B), and 93 human volunteers on Cape Cod (FIG. 9C).

FIGS. 10A and 10B show the exhaled aerosol numbers for 20 subjects 15 minutes after being administered Composition A (FIG. 10A), and several hours after being administered Composition A.

FIGS. 10C and 10D show the suppression effect following a Composition A administration, versus the nasal saline control on overall exhaled aerosol of all 40 subjects at two hours post dosing.

FIGS. 11A, 11B, and 11C compare the effectiveness of nasal saline airway hygiene versus Composition A, shown in exhaled aerosol from all subjects before and 15 to 30 minutes post administration of Composition A or Simply Saline. The results for the 20% highest emitting aerosol subjects are shown in FIGS. 11A-C (Composition A).

FIGS. 12A, 12B and 12C present the overall degree of suppression of exhaled aerosol at each site for both Composition A and Simply Saline at 15 to 20 minutes post administration. Overall airway cleansing by the Simply Saline control is insignificant in every case (BBH p<0.94, GRCC p<0.83, CCA p<0.65), while the overall Composition A airway cleansing effect is marginally significant at each site of the study (BBH p<0.169, GRCC p<0.124, CCA p<0.098).

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with microcontrollers, piezo-electric devices, Peltier devices, power supplies such as DC/DC converters, wireless radios (i.e., transmitters, receivers or transceivers), computing systems including client and server computing systems, and networks (e.g., cellular, packet switched), as well as other communications channels, have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The headings and Abstract of the Disclosure provided herein are for convenience only and do not interpret the scope or meaning of the embodiments.

In particular, described herein are new compositions, systems, methods, and articles of manufacture to advantageously delivery of one or more salt-based formulations or compositions, and in particular physiological salt formulations or compositions that are rich in calcium, to the upper airways of a respiratory tract via a nose to reduce virus shedding and/or as an antimicrobial and/or anti-contagion that is effective against airborne pathogens.

The nasal administration of physiological salts appears particularly effective at reducing airborne particles from exhaled breath including the sub-micron aerosolized particles that are ineffectively filtered by cloth face masks. For example, nasal application of a drug-free, calcium-enriched nasal salt, interacts with airway lining mucus to cleanse the airways of bioaerosols which may reduce exhaled aerosol particles up to 99%, with an overall reduction of exhaled particles in a largest cohort of human subjects of around 75%. Nasal application of physiological salts (e.g., calcium chloride; combinations of calcium chloride and sodium chloride) may, for example, restore the natural surface viscoelasticity of airway lining mucus, reducing the breakup of mucus caused by airflow during natural breathing. As most of these airway droplets are submicron, and as acknowledged by WHO and CDC as potential carriers of SARS CoV-2, their reduction lowers risk of deep lung infection and spread of disease. The nasal administration of physiological salts can be an important addition to current COVID-19 hygiene protocols of mask wearing, hand washing, and social distancing. The nasal administration of physiological salts adds to the efficacy of masks at reducing the penetration of respiratory droplets into the lungs or back into the environment; and provides an added layer of defense for when mask wearing is not a possible.

In particular, a mister or nebulizer that delivers to the site of respiratory droplet formation an aerosol that has a high concentration of calcium may be particularly effective. The ions of the calcium rich salt may associate with mucins on the surface of the airway lining mucus, strengthening resistance to the breakup of mucus. This may advantageously clean the airways of the respiratory droplets that can carry infection and insoluble environmental contaminants.

A new therapeutic practice of nasal application of a calcium-rich saline aerosol to the upper respiratory tract is proposed. Nasal application of physiological salts, and in particular application of an aerosol of droplets containing calcium rich salts (e.g., calcium chloride; combinations of calcium chloride and sodium chloride) in sizes that constrain the aerosol predominately in the upper respiratory tract may produce advantageous antimicrobial effects.

In particular, a mister or nebulizer that delivers to the upper respiratory tract an aerosol that has a high concentration of calcium may be particularly effective. The mister or nebulizer may deliver the calcium rich aerosol with or without other salts that are effective antimicrobials and/or with or without other antimicrobial substances.

These salt-based hygienic and/or antimicrobial formulations or compositions are advantageously formulated as or in readily-soluble water droplets. The readily-soluble water droplets have a median size range of approximately 7 microns to approximately 15 microns, and preferably approximately 10 microns. Thus, the readily-soluble water droplets are too large for significant penetration into the lungs, while being small enough to be carried into the nose and the upper airway of the respiratory tract. Advantageously, such can cause the salt-based hygienic and/or antimicrobial formulations or compositions to be delivered to the upper respiratory track without any appreciable delivery to the lower respiratory track, unexpectedly successfully suppressing or otherwise significantly reducing the shedding of virus. Such can be employed to suppress virus shedding in humans and other animals. Such can additionally or alternatively be employed to produce other beneficial physiological effects.

The droplets are delivered as aerosol, and for instance taken in by the subject through the act of inspiration. The aerosol can be dispensed in an unenclosed volume for instance the open air (e.g., in a room, out of doors), preferably reasonably proximate to a location of one or more individual's faces (e.g., positioned relatively in front of a nose of one or more subjects), without the use of an enclosed volume (e.g., mask, chimney, tumbler, vial, beaker or other container or vessel). Alternatively, the aerosol may be dispensed in an enclosed or partially enclosed volume (e.g., mask, chimney, tumbler, vial, beaker or other container or vessel). The individual(s) draw the salt-based hygienic and/or antimicrobial active substances into the upper respiratory tract via the nose, for instance ortho-nasally and/or retro-nasally, and possibly exhale the hygienic and/or antimicrobial active substances retro-nasally. Open air dispensing may be particularly suitable for treating large crowds, for example crowds entering a stadium or other venue, for instance without the individuals in the crowd touching any objects (e.g., delivery devices or dispensers).

The salt-based hygienic and/or antimicrobial active substance loaded droplets described here may for example, be produced from a small reservoir of less than 100 ML, for instance for individual treatment of subjects. Alternatively, larger reservoirs may be employed, for instance when treating crowds of individuals entering a stadium or other venue or event location.

The salt-based hygienic and/or antimicrobial compositions and formulations may comprise one, two, or more forms of physiological salts, at least one of which is calcium chloride, which are dissolved in water. The salt-based hygienic and/or antimicrobial compositions and formulations may, for example comprise a combination of calcium chloride and sodium chloride, for example in ratio: a 4×isotonic composition (4.72% CaCl₂) in 0.31% NaCl) [0.43M CaCl₂), 0.05M NaCl]; or a 2×isotonic composition (1.29 CaCl₂) in 0.9% NaCl) [0.12M CaCl₂), 0.15M NaCl].

The salt-based hygienic and/or antimicrobial compositions and formulations may include one or more other salts, for instance potassium chloride and/or magnesium chloride. The salt-based hygienic and/or antimicrobial compositions and formulations may include one or more essential oil, fragrance oil or flavor extract (e.g., cacao oil, caramel oil, cinnamon bark oil, coffee oil, eucalyptus oil, palm oil, fig oil, grapefruit oil, hazelnut oil, honeydew melon oil, lavender or spike lavender oil, lemongrass oil, lime oil, black or green pepper oil, peppermint oil, rosemary oil, strawberry oil, smoke oil, tobacco vanilla oil, vanilla oil, chocolate extract, anise extract, rose, and/or linalool) and/or solvents (e.g., ethanol) in addition to water.

Exhaled aerosol numbers appear to be not only an indicator of disease progression, but a marker of disease risk in non-infected individuals. Monitoring exhaled aerosol may be performed as a diagnostic technique in the identification and control of transmission and infection of COVID-19 and other respiratory infectious diseases, including influenza.

A New Natural Defense Against Airborne Pathogens

### INTRODUCTION

Airborne transmission of infectious disease by the very small droplets emitted from human airways on natural breathing, and that accumulate in poorly ventilated indoor environments, has been observed for a range of respiratory diseases, including tuberculosis, measles, chicken pox, influenza and SARS (Yi et al 2007). Recent observations (Zhang et al 2020) place the novel coronavirus SARS-CoV-2—carried by the small airborne droplets exhaled by COVID-19 infected individuals (Lou 2020) and reported stable in aerosol form (Van Doremalen et al 2020) for beyond 3 hours—in the family of airborne transmitted infectious diseases as well. The assessment of SARS-CoV-2 as an airborne pathogen clarifies the nature of the fight against the COVID-19 pandemic (Fauci et al 2020).

While major international scientific efforts advance toward the development of drugs and vaccines in response to the COVID-19 pandemic, less attention has been given to new ways to prophylactically combat airborne viral and bacterial threats. Social distancing, the washing of hands and the wearing of face masks, while each effective and necessary, do not prevent the transmission of pathogens that travel through the air within droplets smaller than 1 μm in diameter. Such small particles are not only too small to be filtered effectively by conventional masks (Bunyan et al 2013, Leung et al 2020, Zhang et al 2020), they are also too small to settle by gravity within the 2 meter threshold of social distancing.

Sub-micron droplets happen to be the majority of particles emit from mouths and noses when breathing naturally. The sub-micron particles emerge from the respiratory system either by the necking of airway lining fluid that occurs with the expansion and contraction of the lungs (Scheuch 2020), or by the rapid movement of air through upper airways, as occurs during natural breathing, coughing, sneezing, and speaking (Watanabe et al 2007). Whether high or low in the airways, shedding of small droplets from infected lungs can carry viral and bacterial pathogens into the environment, promoting disease spread (Leung et al 2020, Edwards et al 2004). When this shedding occurs in the upper airways, it can promote movement of pathogen deeper into the lungs, and self-infection.

The delivery of saline to the respiratory system has been observed to diminish the exhalation of these very small particles (Edwards et al 2004). This diminution is due to electrostatic interactions between salt cations and mucin and mucin-like proteins on the surface of airway lining fluid. These increase the viscoelasticity of the surface of airway lining fluids (Watanabe et al 2007), reducing the formation of airborne droplets (Watanabe et al 2007). Calcium chloride, far more than sodium chloride, has been found to increase the surface elasticity of airway lining fluid, potentially promoting even more substantial suppression of airborne particle generation in the airways, while enhanced surface plasticization also further resists pathogen penetration through the mucus layer, strengthening its biophysical barrier to infection (Watanabe et al 2007).

Calcium and sodium salts appear to have antimicrobial properties as well. High concentrations of extracellular calcium (Krisanaprakornkit et al 2003), as can be achieved by the aerosol delivery of calcium salts, promote the secretion of β-defensin 2 from nasal epithelial cells (Alp et al 2005). Human β-defensin 2 is an endogenous antimicrobial peptide that conjugates with receptor-binding domains of many viruses, including coronaviruses, to promote expression of antiviral and immune-inducing molecules as well as chemokine recruiters of leukocytes (Kim et al 2018). Human β-defensin 2 has been shown to be effective as an antiviral adjuvant due to its binding to the spike protein of MERS CoV (Zhao et al 2016), and mouse beta defensin 4 derived peptide has shown activity against SARS CoV-1 (Kim et al 2018).

Chloride salts have been shown to diminish viral replication as far back as the 1960s (Speir 1961). Chloride ions promote antiviral activity by the induction within cells of hypochlorous acid, the active constituent of bleach (Ramalingam et al 2018). Chloride salts induce innate immune response of epithelial cells in the presence of sodium chloride (Ramalingam et al 2018). Sodium hypochlorite, the sodium salt of hypochlorous acid, has particularly demonstrated effectiveness as a disinfectant against coronavirus (Geller et al 2012, Dellanno et al 2009). High concentrations of chloride, delivered via hypertonic saline to nasal epithelial tissues, have been found to diminish viral infections associated with the common cold (Ramalingam et al 2019, Adam et al 1998, Šlapak et al 2008). In an open-labeled randomized controlled human study of 68 subjects with common cold infections including rhinoviruses and coronaviruses, as well as enterovirus, influenza A virus, nasal delivery of 2-3% hypertonic saline 2-8 times a day (median thrice-a-day) significantly lowered duration of illness, as well as use of over-the-counter medications, household transmissions, and viral shedding (Ramalingam et al 2019). Nasal administration of 3.5% hypertonic seawater has similarly shown indications of efficacy against common cold symptoms in other human clinical trials (Adam et al 1998, Šlapak et al 2008).

Described herein are various salt compositions incorporating at least two ions that are abundant in human tissues: calcium, and chloride, and optional a third ion sodium, which may be used for hygienic applications, for example to address the need for a broad prophylactic and anti-contagion defense against respiratory viral and bacterial infections. Without being bound by theory, the inventor(s) hypothesized that an aerosol combining calcium and optionally sodium salts would improve the barrier function of the mucus lining to protect against infection while diminishing bioaerosol formation in the lungs and nasal passages. Given the hygienic practice of nasal saline flushing, the nasal delivery of these physiological salines were evaluated with a specially designed nasal mist, as a practical, efficacious and safe personal hygiene intervention, complementary to masks. This might prove of particular utility to the immediate fight against the current COVID-19 pandemic.

Results

Nasal Delivery Device

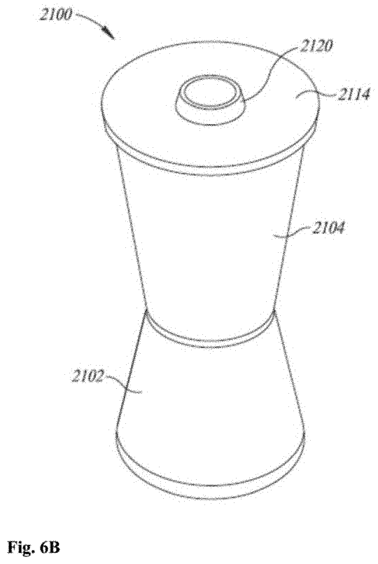

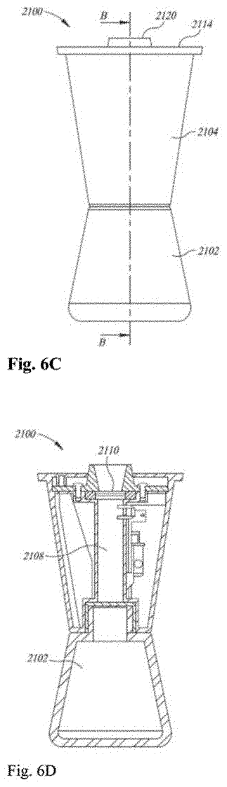

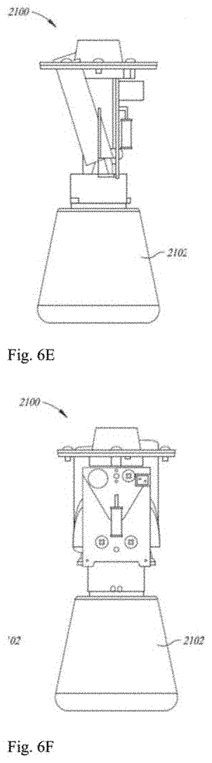

A hand-held nebulizer (Nimbus™) device was designed capable of delivering salt-based hygienic and/or antimicrobial formulations or compositions nasal doses of around 1-2 mg CalCl₂. The nebulizer device employed integrated vibrating meshes with a 6 μm pore size to produce, on tipping of the device, an aerosol cloud with a particle size distribution optimal for delivery within the nose through natural nasal inspiration. The particle size distribution of the aerosol cloud reveals a median volume particle diameter of 9-10 μm, an optimal size for nasal and upper airway deposition of aerosol following a natural tidal inspiration through the nose and with relatively uniform distribution of deposition from the anterior to the posterior of the nose (Calmet et al 2019). The particle size distribution is significantly smaller than that produced from a standard nasal pump spray (FIG. 6B).

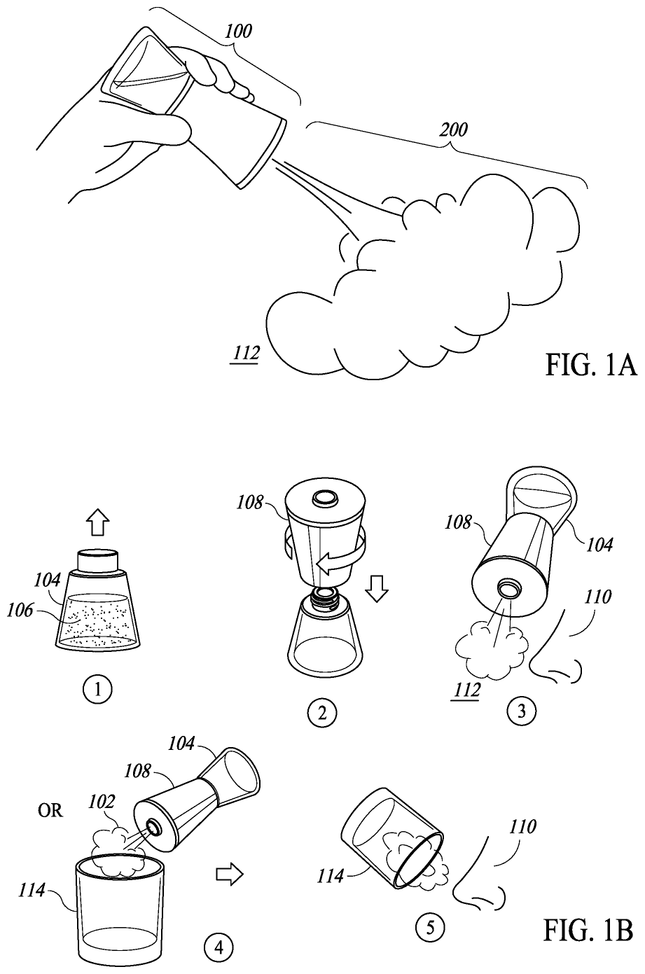

On tipping (FIG. 1A), Nimbus™ nebulizer device produces 57 mg+/−2 mg within a 10 second actuation, after which power ceases until tipped back upright and again overturned. The Nimbus™ nebulizer device is designed to deliver a controlled dose of approximately 33 mg (i.e., 1.56 mg CalCl₂ or 0.43 mg CalCl₂) by filling an empty 6 oz. glass with the cloud for the internally programmed 10 second actuation of the device and then inspiring the cloud directly from the glass into the nose (FIG. 1B). Uncontrolled dosing can also be achieved by creating the cloud before the nose and direct natural deep nasal inspiration (FIG. 1B).

We decided to pursue a human volunteer study with Nimbus™ nebulizer device to evaluate the effectiveness of salt-based hygienic and/or antimicrobial formulations or compositions for suppressing exhaled aerosol particles following nasal administration in comparison to our observation of the effectiveness of the salt-based hygienic and/or antimicrobial formulations or compositions on pulmonary delivery.

Human Exhaled Bioaerosol Studies: Nasal Administration

Ten healthy volunteers were recruited in St Augustine, Fla. and Boston, Mass. Each signed informed consent to participate in the several-hour nasal saline hygiene study. Five of these subjects were older than 65 (70, 75, 82, 83, 88) and five younger than 65 (30, 40, 59, 60, 63). Subjects with severe respiratory illnesses were excluded from the study, while two of the subjects (ages 30 and 63) were cigarette smokers.

All subjects began the study by breathing into an apparatus that measured expired aerosols. Following a baseline assessment of exhaled aerosol particle count subjects drew two deep nasal inspirations of an 4×isotonic composition of 1.29% CaCl₂ and 0.31% NaCl dissolved in water via Nimbus™ nebulizer device. Subsequent to administration, subjects breathed into the airborne particle detector at intervals for up to 6 hours post-dosing. Subjects also self-administered a commercial (isotonic sodium chloride) simple saline cleansing spray (CVS Nasal Saline). Subsequent to nasal administration of the placebo control, subjects breathed into the airborne particle detector at intervals for up to 2 hours.

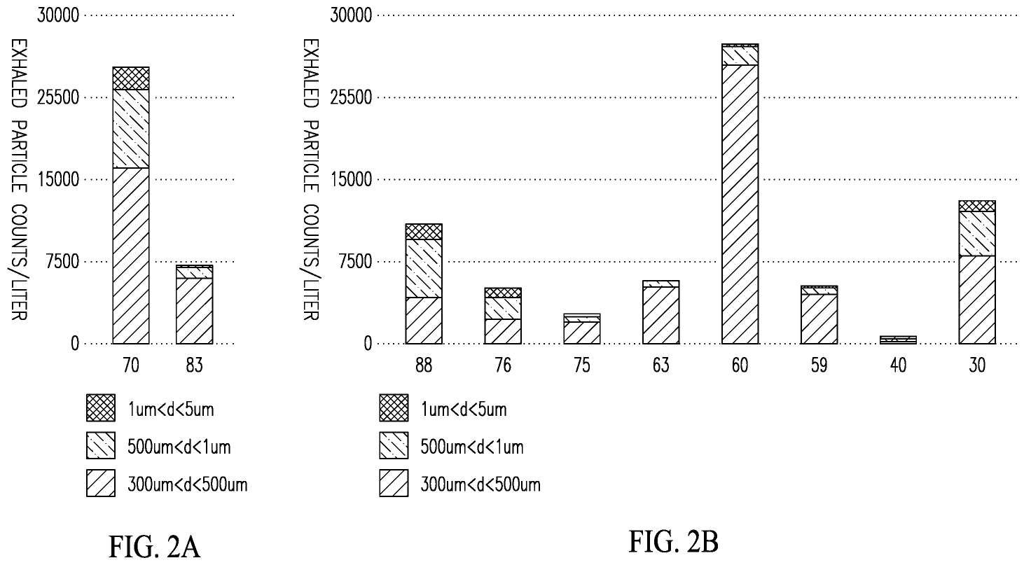

Baseline exhaled particles per liter per subject age are shown in FIGS. 2A and 2B. Two of the ten subjects in the older age (>65) group exhaled very high numbers of particles per liter of air (24,088+/−9,413 and 7,180+/−1250) (FIG. 2A) while the other eight (FIG. 2B) exhaled between approximately 10 and 1200 particles per liter. In the latter group two individuals (ages 30 and 63), were smokers. There is a strong correlation between high numbers of exhaled particles and age, with the group older than 65 exhaling on average 6,641 particles per liter while the group younger than 65 on average 440 particles per liter. In all subjects over 95% of the baseline exhaled particles were less than 1 micrometer in size, with most smaller than 500 nm.

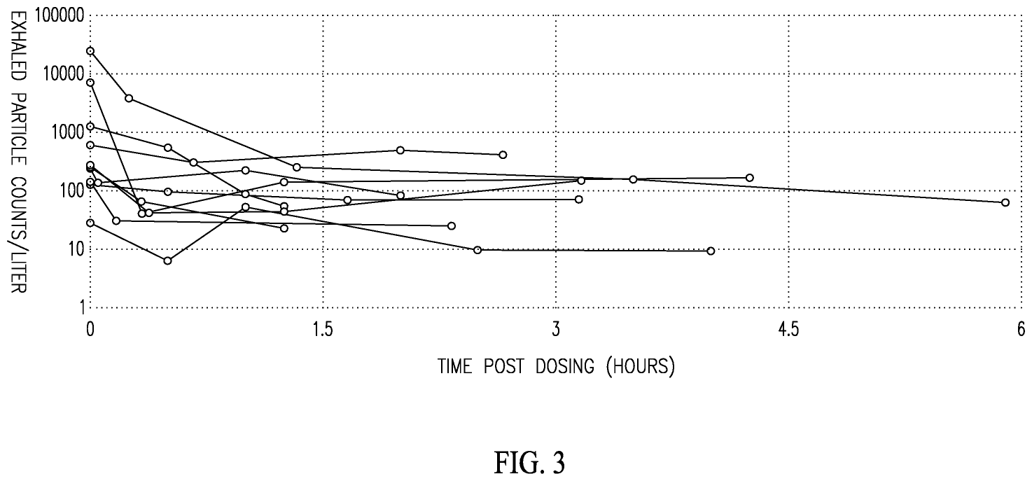

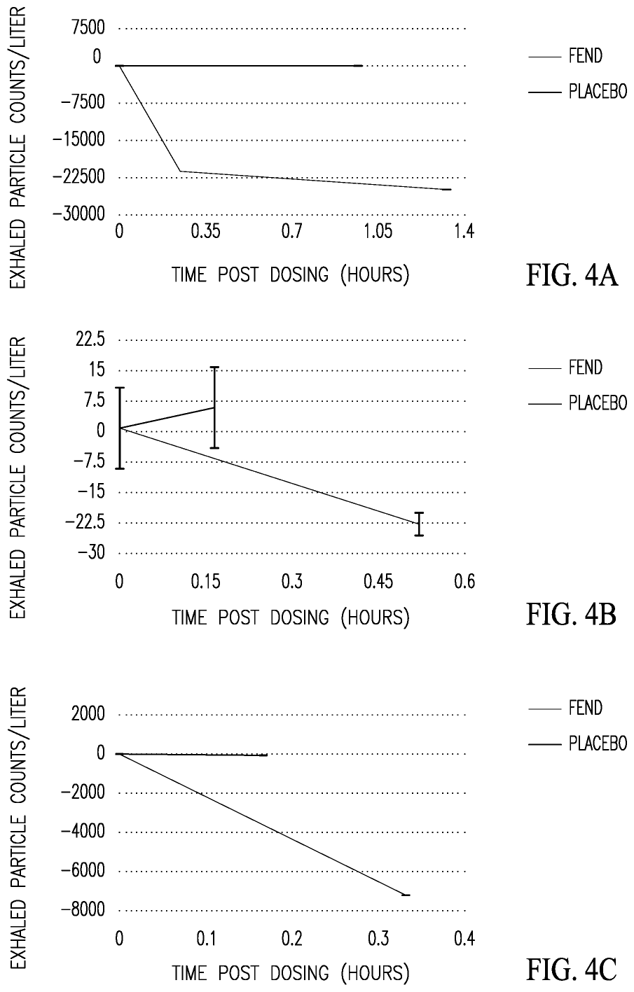

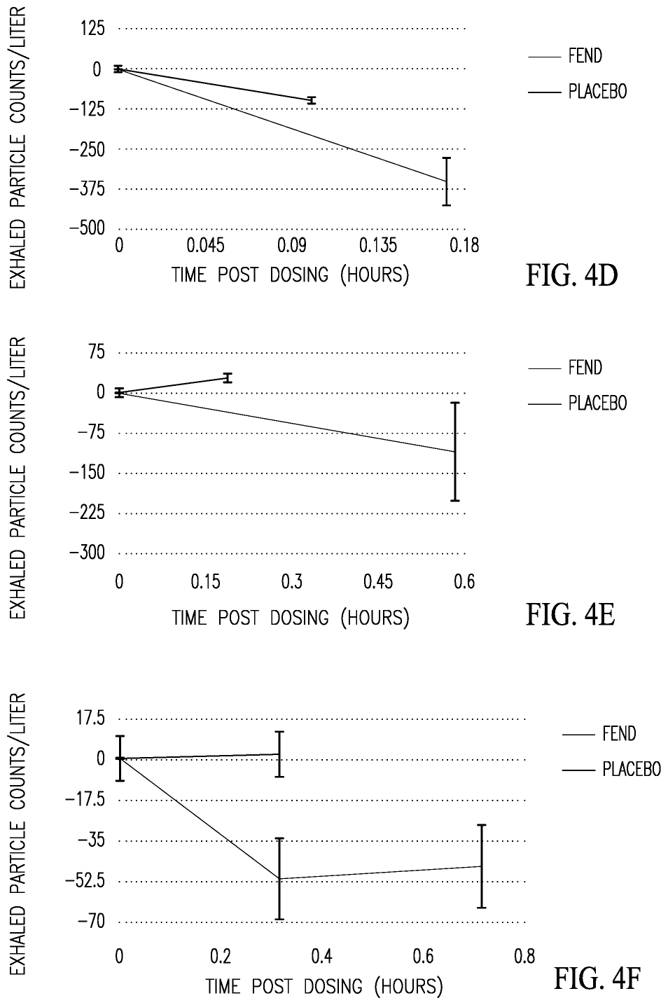

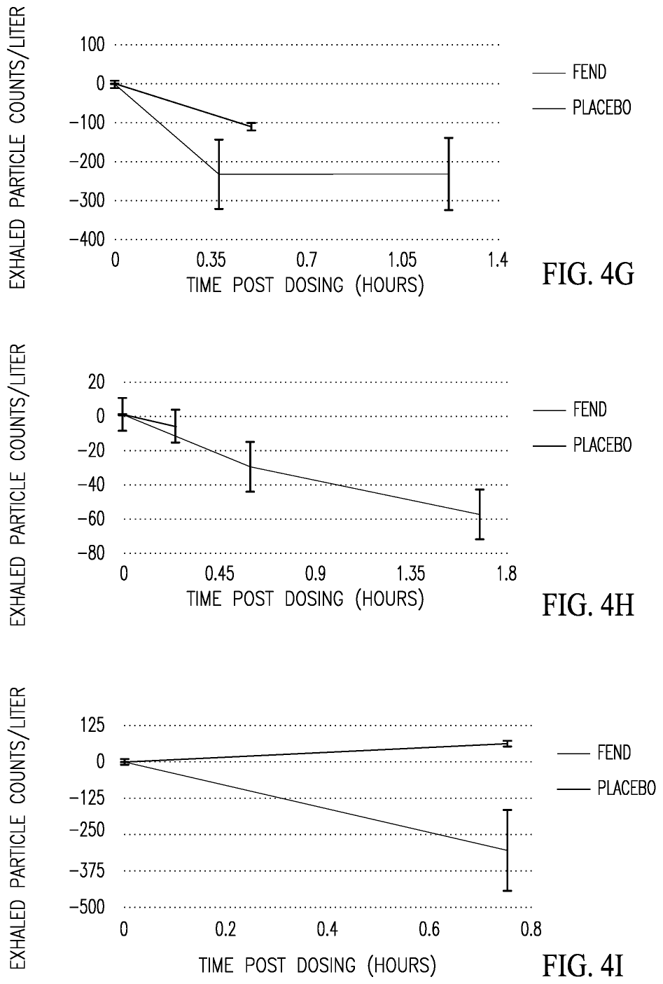

Following administration of the salt-based hygienic and/or antimicrobial formulation or composition, exhaled particle numbers diminished for up to several hours as shown in FIG. 3. This diminution relative to baseline is statistically significant (p<0.05) for all 10 subjects. Duration of effect continued up to the last data point several (2-6) hours after administration for all of the subjects other than subjects B and E, each of whom were very small producers of particles. Administration of the simple saline control has a minor suppressive effect on exhaled particles for 2 of the subjects in the first hour following administration while in the other subjects we observed no suppressive effect (FIGS. 4A-4I).

Using the lowest exhaled particle number following administration as a measure of suppression effect, diminution of bioaerosol ranged from a low of 45% (subject age 75) to a high of 99% (subject ages 83 and 70), with overall suppression of aerosol for the group (99%) predominantly related to the dramatic effect of aerosolized salt-based hygienic and/or antimicrobial formulations or compositions on suppression amongst super producing individuals (subject ages 83 and 70).

### Discussion

Hypertonic calcium chloride and sodium chloride solution delivered to the respiratory system appears to have potential as both hygienic and therapeutic biodefense against airborne pathogens. Hygienically, these physiological salts coat the surfaces of airway lining fluid to diminish breakup and clear away the sub-micron bioaerosol droplets that are not effectively captured by masks. By potentially boosting natural immunological defenses—strengthening the barrier function of the airway lining fluid and promoting the secretion of β-defensin 2 from nasal and bronchial epithelial tissues—these salts may also act therapeutically for antimicrobial prophylaxis or treatment.

While these results suggest that calcium and sodium chloride salt combinations may be therapeutically useful against bacterial and viral infections including influenza, rhinovirus and pneumonia, these results point to an immediate hygienic value in the use of calcium chloride salt formulations and compositions in the fight against any airborne infectious disease, including COVID-19, by cleaning the airways of the small airborne droplets that carry airborne infection.

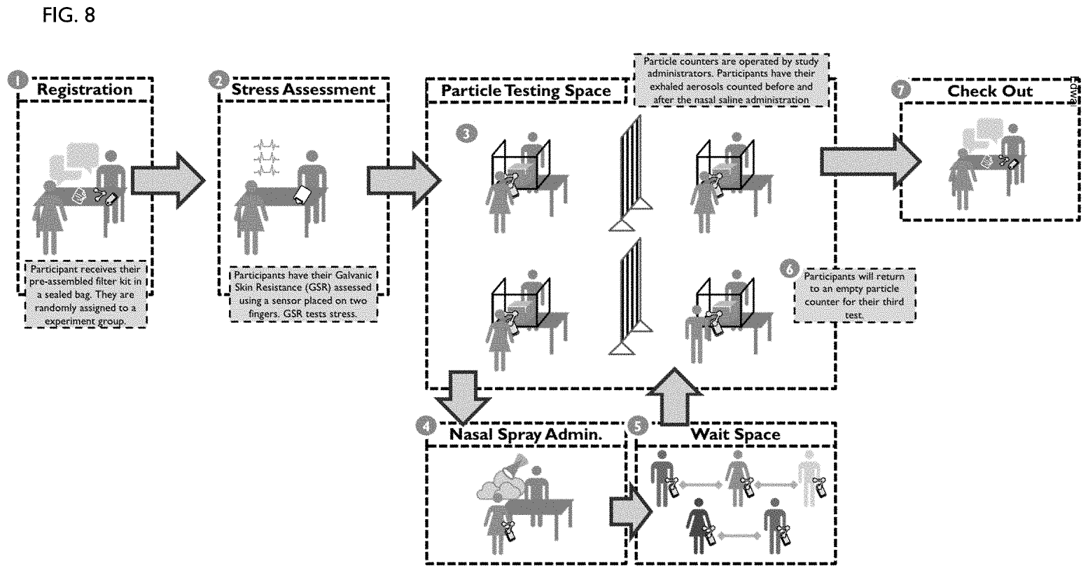

The finding that nasal inspiration of salt-based hygienic and/or antimicrobial formulation or composition of 1.29% CaCl₂ and 0.31% NaCl dissolved in water in a group of 10 healthy human subjects reduces exhaled particles between 45% and 99% by way of an aerosol too large to penetrate the lower airways (FIGS. 8 and 4A-4I), suggests that the upper airways are a primary source of expired bioaerosol. The high velocity airstreams created during natural breathing (often reaching turbulent air flow conditions) in the trachea and main bronchi disturb the surfaces of airway lining fluid in the way of wind passing over the sea to generate sea mist. Such phenomena are highly sensitive to compositional variations in the underlying fluid, making exhaled bioaerosol a sensitive measure of airway lining fluid and introducing variability within and between subjects.

In the study the application of the salt-based hygienic and/or antimicrobial formulation or composition substantially cleared away exhaled particles, most being less than 1 μm in size. That particles in the range of 300 to 500 nm were the most predominant observed in the exhaled breath of subjects can be explained by the fact that such particles are both too small to deposit in the lungs by gravity or inertia, once generated, and too large to be deposit by diffusion. These are the submicron particles most likely to remain suspended in the atmosphere essentially indefinitely. Possibly most important in terms of their ability to transmit infection—and deposit on surfaces including airways of the infected or naive individuals—are those particles in the 500 to 1000 nm range, a significant fraction of the exhaled particles of the super spreader individuals. These particles are also substantially eliminated by the salt-based hygienic and/or antimicrobial formulation or composition treatment.

In the study most of the airborne particles were exhaled from two of ten “super producing” individuals. This super production of bioaerosol promotes the phenomenon of super spreaders (Stein 2015). Super spreading events for COVID-19 have been reported in China (so-called patient 31), India (the Punjab outbreak), South Korea, and many other regions, and are suspected to be a primary mode of transmission of the disease (Kupferschmidt 2020). Among key correlates of super spreading are suppressed immunity and infected lungs (Stein 2015)—two particular vulnerabilities of the most aged. Indeed older subjects in our study exhaled many more particles than younger subjects—suggesting the possibility that seniors, while among the most vulnerable to COVID-19 infection, may also be those most likely to spread the disease, and underscoring the extreme risk seniors face today in nursing homes.

As nasal hygiene, a salt-based hygienic and/or antimicrobial formulation or composition is easy to administer (FIG. 1B), rapid (one or two deep nasal inspirations) and lasts long (at least 6 hours in those expiring the largest numbers of particles). It might be easily administered to individuals on entering environments where they are likely to encounter others, including hospitals, nursing homes, prisons, schools, offices, factories, stadia, restaurants, and museums. The use of salt-based hygienic and/or antimicrobial formulations or compositions as an “invisible mask” supplement to traditional masks administered prior to close encounters with others in public and private spaces in order to clean the air of the small particles that masks do not block appears a prudent addition to current hygienic practices in the face of the COVID-19 pandemic.

More research may be performed to assess the consequences of calcium-enriched physiological salt nasal hygiene on airborne infection and transmission rates within environments at high-risk of COVID-19 and other airborne infectious diseases. The therapeutic potential of these nasally and pulmonary delivery salts should also be explored beyond the scope of the in vitro and animal a studies reported here.

FIG. 1A is an isometric view of a delivery device 100 delivering an aerosol 102 of a salt-based hygienic and/or antimicrobial formulation or composition into an unconfined space or volume to be inspired by a subject, according to at least one illustrated implementation. Tipping of a Nimbus™ nebulizer device with respect to a gravitational axis of the Earth actuated an actuator to cause a mesh to vibrate, thereby generating an aerosol cloud for dosing.

FIG. 1B is an illustrative diagram of various sequential acts performed in using a delivery device to deliver an aerosol of a salt-based hygienic and/or antimicrobial formulation or into either a unconfined or free space or volume, or into a confined space or volume (e.g., mask, chimney, tumbler, vial, beaker or other container or vessel), to be inspired by a subject, according to at least one illustrated implementation. A salt-based hygienic and/or antimicrobial formulation or composition can be administered by a Nimbus™ nebulizer device with a deep nasal inspiration either in an unconstrained environment, for instance before the nose of a subject, or in a constrained environment (e.g., by containing the aerosol cloud in a partially enclosed environment such as a mask, chimney, tumbler, vial, beaker or other container or vessel).

As illustrated in FIG. 1B, a reservoir 104 containing the salt-based hygienic and/or antimicrobial formulation or composition 106 (e.g., CaCl₂)) dissolved in water (e.g., distilled water) is provided at 1. At 2, the reservoir 104 is then coupled to a dispenser portion 108 of the Nimbus™ nebulizer device in a generally upright (with respect to the gravitational axis) orientation. The dispenser portion 108 of Nimbus™ nebulizer device 100 may include a housing, a mesh, an actuator coupled to drivingly oscillate the mesh at a desired frequency, and/or drive circuitry. The drive circuitry may, for example, include an accelerometer, geomagnetic field sensor, level sensor, and/or gyroscope, which activate the actuator on sensing a tipping of the Nimbus™ nebulizer device relative to the gravitational axis. The drive circuitry may include a timer, that deactivate the actuator after a defined period of time, for example, to control the dosage of the salt-based hygienic and/or antimicrobial formulation or composition dispensed.

At 3, the Nimbus™ nebulizer device 100 is positioned proximate the face and/or nose 110 of a subject, and tipped relative to the gravitational axis to dispense a salt-based hygienic and/or antimicrobial formulation or composition as an aerosol 102 into an unconstrained or free space or volume 112, not contained by an enclosure. In response, the drive circuitry activates the actuator to vibrate the mesh, causing the salt-based hygienic and/or antimicrobial formulation or composition to be dispensed as an aerosol proximate the nose 110 of the subject, for inspiration by the subject via the nose 110 (e.g., ortho-nasally) and into the upper airways of the respiratory tract.

Alternatively, at 4 the Nimbus™ nebulizer device 100 is positioned proximate an opening of a container or vessel 114, and tipped relative to the gravitational axis to dispense a salt-based hygienic and/or antimicrobial formulation or composition as an aerosol 102 into a constrained or at least partially enclosed space or volume, at least partially contained by an enclosure (e.g., mask, chimney, tumbler, vial, beaker or other container or vessel 114). In response, the drive circuitry activates the actuator to vibrate the mesh, causing the salt-based hygienic and/or antimicrobial formulation or composition to be dispensed as an aerosol 102 into the container or vessel 114. At 5, the container or vessel 114 is positioned proximate the face and/or nose 110 of a subject, for inspiration by the subject via the nose 110 (e.g., ortho-nasally and/or retro-nasally) and into the upper airways of the respiratory tract.

As used herein a confined or partially confined or constrained volume refers to a vessel sized volume (e.g., on the order of 1 foot³ or approximately 28316 cm³) as compared unconfined or unconstrained volumes (e.g., room sized volumes on the order of 100 foot³ or approximately 2.8 m³).

FIGS. 2A and 2B show a measure of exhaled particles from ten (10) human volunteers prior to salt-based hygienic and/or antimicrobial dosing. The exhaled particles per liter of air are shown within three size distributions, between 300 and 500 nm, between 500 nm and 1000 nm, and between 1000 nm and 5000 nm. In particular, FIG. 2A shows results from two (2) of the human subjects (ages 63 and 70) who exhaled greater than 25,000 and 7000 particle per liter respectively, the majority of these particles between 300 and 500 nm, and a large minority of the particles between 500 nm and 1000 nm. FIG. 2B shows the results from the other eight (8) individuals who breathed out on average several hundred particles per liter.

FIG. 3 shows the measure of exhaled particles from each of the ten (10) human volunteers following salt-based hygienic and/or antimicrobial dosing. In all cases statistically significant suppression of exhaled aerosol is observed while the effect is dramatically significant for the largest “super producing” subjects (ages 63 and 70), whose overall exhaled particle counts diminish more than 99% for 6 h following salt-based hygienic and/or antimicrobial nasal inspiration.

FIGS. 4A-4I are graphs showing measures of exhaled particles per subject following salt-based hygienic and/or antimicrobial dosing in comparison to the placebo control. All exhaled particles per liter (all sizes) are shown with standard error bars up to one hour post dosing comparing the effects of salt-based hygienic and/or antimicrobial formulation or composition and isotonic saline (CVS Saline Spray) dosing on expired aerosol numbers In particular, for the subjects represented in FIGS. 4D and 4G, the saline control shows significant suppression, while for the subject represented in FIG. 4F, it shows significant amplification. In all cases, aerosolized treatment with a salt-based hygienic and/or antimicrobial formulation or composition suppresses exhaled aerosol counts relative to the control (p<0.05) when comparisons are made between the closest time points of counts measured. The ages of the human subjects shown are: (A) 83 (B) 40 (C) 70 D) 88 (E) 76 (F) 59 (G) 63 (H) 75 (I) 30.

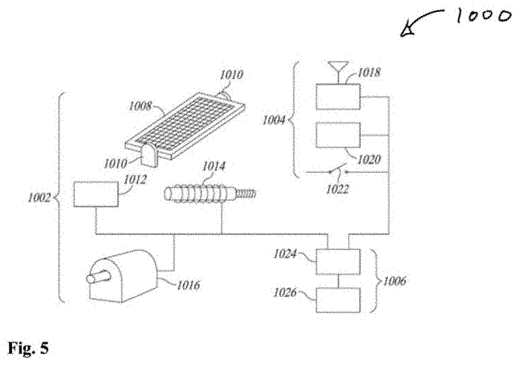

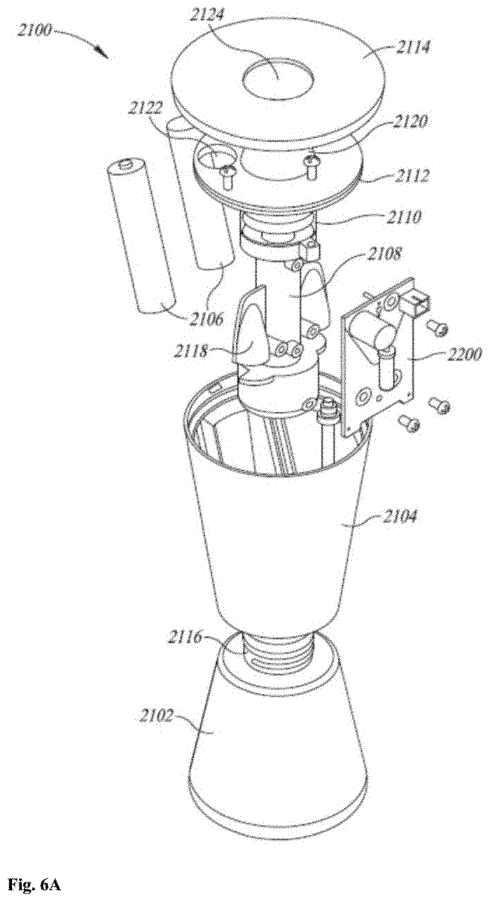

FIG. 5 is a schematic diagram that shows a portion of a nebulizer delivery device 1000 according to at least one illustrated implementation. The nebulizer delivery device 1000 may take the form of, or otherwise include, a nebulizer 1002, with one or more actuators 1004, and a control subsystem 1006 and, or other electronics, according to at least one illustrated implementation.

The nebulizer 1002 can include one or more mesh screens 1008, for example a metal mesh screen, which is supported by a frame 1010 for movement, for example for oscillation or rotation The nebulizer 1002 can include one or more of a piezo-electric element 1012, solenoid 1014 or electric motor 1016 physically coupled to move the mesh screen(s) 1008 along at least one axis in response to signals from the microcontroller to dispense aerosol into the chamber. In some implementations, the actuator is physically coupled to the mesh screen 1008 via one or more mechanical transmissions (e.g., elliptical gear) or magnetic transmissions. The nebulizer may, for example, oscillate the screen at ultrasonic frequencies to cause a dispersion of the scent media. The transducer may oscillate at a frequency of about 175 kHz±5 kHz that is sufficient to atomize the fluid held in the fluid reservoir. The frequency of oscillation of such a transducer may be increased or decreased depending up on the properties of the fluid or other materials held within the fluid reservoir. In such an implementation, that transducer may form an annular ring with a metal-mesh included within a center portion of the transducer. In some implementations, the metal-mesh screen 1008 may be fluidly coupled to the fluid reservoir via capillaries, thereby providing a fluid path that enables a low flow of the fluid from the fluid reservoir to the metal-mesh screen 1008. As such, the fluid may be transported to the metal mesh, via, for example, capillary action, where it is atomized into the vapor or aerosol as a result of the oscillation of the transducer. In some implementations, the metal-mesh screen 1008 may provide a filter that prevents large sized molecules from being emitted as part of the vapor or aerosol that exits the dispenser. As such, the metal-mesh screen 1008 may have mesh openings that are 500 micrometers in width. In some implementations, the mesh openings may be less than 500 micrometers in width (e.g., 100 micrometers, 200 micrometers, 300 micrometers, or 400 micrometers). Preventing the larger molecules from being dispensed may provide for a better user experience by reducing the possibility that the vapor or aerosol will irritate the user.

The nebulizer 1002 may include one or more of radios 1018, transducers or sensors 1020 and, or, switches 1022 communicatively coupled to the control subsystem 1006.

The control subsystem 1006 may, for example, include one or more microcontrollers 1024, microprocessors, field programmable gate arrays, and, or application specific integrated circuits. The control subsystem 1006 may, for example, include one or more nontransitory storage media 1026 that stores at least one of processor-executable instructions or data, which when executed by the microcontroller 1024 causes the microcontroller 1024 to control operation of the device 900, for example in response to one or more inputs. For example, the microcontroller may receive signals from one or more of radios 1018, transducers or sensors 1020 and, or, switches 1022, and control operation of the nebulizer 1002 in response to same. For instance, the control subsystem may cause the nebulizer to dispense or disperse scent media in response to a first input, and to stop the nebulizer 1002 from dispensing or dispersing salt-based antimicrobial and/or anti-contagion formulations or compositions in response to a second input. Input can include user manipulation of a switch, positioning or orientation of the vessel by the user, or wireless commands from a radio or remote controller.

The nebulizer delivery device 1000 may, for example, include one or more switches and/or sensors. The switch(es) and/or sensor(s) may be communicatively coupled to the microcontroller and operable to produce a signal that causes the microcontroller to operate the actuator accordingly. The switches may, for instance, include one or more of any of the following: a contact switch, a momentary contact switch, a rocker switch, etc. The sensors may, for instance, include one or more of any of the following: The device may, for example, include one or more sensors, for instance a one-, two- or three-axis accelerometer, a PIR motion sensor, an inductive sensor, a capacitive sensor, and, or Reed switches. The switch(es) and/or sensor(s) may, for example, be operable to produce a signal that causes the microcontroller to operate the actuator in response to the at least one nebulizer delivery device 106 being coupled to at least one of the docks. The switch(nebulizer) and/or sensor(s) may, for example, be responsive to a presence or an absence of the vessel with respect to a base and operable to produce a signal that causes the microcontroller to operate the actuator according to the presence or an absence of the vessel with respect to the base. The switch(es) and/or sensor(s) may, for example, be responsive to a position or orientation of the vessel and operable to produce a signal that causes the microcontroller to operate the actuator according to the orientation of the vessel. The switch(es) and/or sensor(s) may, for example, be part of the at least one nebulizer delivery device.

The nebulizer delivery device 1000 may include a transducer communicatively coupled to operate the nebulizer. The transducer may, for example, include one or more radios (e.g., cellular transceiver, WI-FI transceiver, Bluetooth transceiver) which receives wireless signals for instance RF or microwave signals for one or more wireless communications devices (e.g., smartphones) or remote controllers. The transducer may, for example, include one or more receivers, for instance an infrared receiver that receivers infrared light signals from a remote controller.

Activation may be synchronized with the delivery of audio, video, or audiovisual media. For example, a smartphone or digital assistance (e.g., Amazon Alexa®, Google Home®, Apple HomePod®) can cause activation of nebulizer 1002.

A suitable microcontroller may take the form of an 8-bit microcontroller with in-system programmable flash memory, such as the microcontroller commercially available from Atmel Corporation under designation ATMEGA48/88/168-AU. The microcontroller executes a program stored in its memory, and sends signals to control the various other components, such as, for example, the valves. Control signals may, for instance be pulse width modulated (PWM) control signal, particularly where controlling an active power supply device. Otherwise, control signals may take on any of a large variety of forms. For instance, the microcontroller may operate valves or the actuator 1004 simply by completing a circuit that powers the respective value or actuator 1004.

The nebulizer delivery device 1000 may optionally include a visual indicator (not illustrated) to indicate when the nebulizer delivery device 1000 is operating or turned ON. Although a single light emitting diode (LED) may be employed, the visual indicator may take any of a large variety of forms. The LED may be capable of emitting one, two or more nebulizer colors. The visual indicator may also indicate other information or conditions, for instance the visual indicator may flash in response to an occurrence of an error condition. A pattern of flashes (e.g., number of sequential flashes, color of flashes, number and color of sequential flashes) may be used to indicate which of a number of possible error conditions has occurred.

In some implementations, the nebulizer delivery device 1000 is electrically powered by one or more batteries that may provide a power source for the oscillation of the actuator 1004. The battery may be small and lightweight, such as the batteries used for small electronic devices (e.g., hearing aids). In some implementations, the battery is at least partially embedded within the nebulizer delivery device. In some implementations, the battery is selectively removable and replaceable, such as when the battery can no longer provide sufficient charge to operate the nebulizer delivery device 106. Other types of power sources may be provided, such as a power source comprised of one or more photovoltaic panels and associated components that may convert light into energy that can be used to operate the nebulizer delivery device 106, an array of super- or ultra-capacitor cells, or an array of fuel cells.

While not illustrated, one or more cartridges may carry the salt-based antimicrobial and/or anti-contagion formulation or composition to be dispensed. The cartridges are sized and dimensioned to be removably receivable by a scent media reservoir of a nebulizer delivery device, to supply a solution of the salt-based antimicrobial and/or anti-contagion formulation or composition to the nebulizer for dispersion, for example as a spray of droplets or an aerosol. The cartridges may be made of plastic. Single use cartridges may, for example contain a single dose of the substance to be dispensed, stored in a liquid form. Alternatively, large reservoirs may employed at large venues and events.

The cartridges may form a fluid reservoir and may be comprised of a polymer, elastomer, or other light-weight, durable material that may be used to hold a liquid. The cartridges may be formed of one or more plastics, for example an ABS or polycarbonate plastic. The plastic may be injection molded or vacuum molded to form the cartridges. The type of material or process employed to form the cartridges from the material should not be considered limiting. In some implementations, the cartridges may include an interior cavity that forms the fluid reservoir that may be used to hold and contain one or more salt-based antimicrobial and/or anti-contagion formulations or compositions as a fluid or other material (e.g., powder, gel, colloidal suspension) that carries active substances (e.g., calcium chloride and sodium chloride). In some implementations, for example, the fluid reservoir may be sized and dimensioned to hold up to 100 mL of the fluid. In some implementations, the fluid reservoir may be sized and dimensioned to hold a maximum amount of the fluid that used to form a single dose, which may, for example, hold less than 100 mL (e.g., 5 mL, 10 mL, 20 mL, 40 mL, or 50 mL). The fluid may be any liquid or other material that is, or that carries, the salt-based antimicrobial and/or anti-contagion formulation or composition that are released when the fluid transitions to a vapor or aerosol and is released. The cartridges may include an aperture that forms part of the fluidly communicative path for the fluid to be transferred from the fluid reservoir to the a nebulizer to be converted into a vapor or aerosol. The vapor or aerosol may advantageously comprise readily-soluble water droplets have a median size range of approximately 7 microns to approximately 15 microns, and more preferably about 10 microns. Thus, the readily-soluble water droplets are too large for significant penetration into the lungs, while being small enough to be carried into the upper airways of the respiratory tract via the nose.

FIGS. 6A-6F illustrate various views of a handheld Nimbus™ nebulizer delivery device 2100 for producing and delivering a cloud of vaporized salt-based antimicrobial and/or anti-contagion formulation or composition in aerosol form.

The hand-held nebulizer (Nimbus™) operates on the basis of a vibrating mesh activated by two replaceable AAA batteries. The device is comprised of a head, which contains the piezoelectric vibrating mesh and on/off trigger, and a base or 1 oz. (30 mL) vial into which the salt-based hygienic and/or antimicrobial formulation or composition solution can be filled. The Nimbus™ vial is detachable and made either of glass or plastic, full of sterile solution and discarded once empty. To evaluate delivered dose a 4-place balance (0.1 mg precision) was used along with the hand-held nebulizer. Nimbus™ was inverted and cloud dispensed into a 6 ounce jar covered by a disk with a hole for cloud emission into the glass container. After ten seconds the cloud ceased to form, the Nimbus™ was removed, the disk removed, and the weight of the glass determined. The “Discharged Dose” (n=5) results comprise measuring the entire 10-second emission into the jar through the coaster, and capping the coaster hole immediately after. The total emitted mass from the device was determined to be 57.0±2.1 mg. Approximately 22.1±1.5 of the dose deposited on the walls of the glass or ˜39% of the emitted dose. Nasally delivered dose was assessed by two users affecting a single nasal inhalation from the glass post filling (n=5). The results 22.6 mg and 23.4 mg, respectively suggest a reproducible delivery of the solution and in the range of the target nasal dose.

The device 2100 can include any of the features of any of the other devices described herein, and can be used in combination with any of the other devices described herein. As illustrated in FIG. 6A, delivery device 2100 includes a base 2102, which can be transparent and which includes a hollow container or tank or vial, in some cases having a volume or capacity of less than 100 mL, for holding salt-based antimicrobial and/or anti-contagion formulation or composition in a liquid form. The base 2102 also includes an upwardly-extending hollow conduit, tube, or pipe 2116, through which the salt-based antimicrobial and/or anti-contagion formulation or composition can be poured out of the base 2102 in a liquid form. An exterior surface of the conduit 2116 includes a set of threads.

The delivery device 2100 also includes a top or upper portion or main body 2104, which includes a hollow housing and the electronic and mechanical components of the delivery device 2100. Such components include a printed circuit board 2200 and associated components coupled thereto, a pair of batteries 2106, a hollow conduit, tube, or pipe 2108, a piezo-electric device 2110, which can include or be physically coupled to a mesh screen having a mesh size of 3 microns, of 4 microns, of 6 microns, of 20 microns, or of between 3 and 20 microns, as well as an internal cover 2112, and an external cover 2114, which can be transparent or translucent. The housing of the main body 2104 can be opaque or translucent, and can have a specific color such as red, orange, yellow, green, blue, purple, brown, black, or white. The internal cover 2112 can have an appearance matching that of the housing of the main body 2104. In particular, the internal cover 2112 can be opaque if the housing of the main body 2104 is opaque or translucent if the housing of the main body 2104 is translucent, and can have a specific color matching that of the housing of the main body 2104, such as red, orange, yellow, green, blue, purple, brown, black, or white.

The conduit 2108 includes a relatively wide top end portion, a relatively narrow middle portion and a relatively wide bottom end portion sized to extend around the conduit 2116 of the base 2102. An inner surface of the bottom end portion of the conduit 2108 includes threads complementary to the threads of the conduit 2116 so that the conduits 2108 and 2116 can be threadedly engaged and thereby coupled to one another. When the conduits 2108 and 2116 are coupled to one another, liquid salt-based antimicrobial and/or anti-contagion formulation or composition can be poured out of the base 2102 through the conduit 2116 and into the conduit 2108. The relatively wide top end portion of the conduit 2108 is sized and configured to house the piezo-electric device 2110 at the top end of the conduit 2108, so that the liquid salt-based antimicrobial and/or anti-contagion formulation or composition can flow through the conduit 2108 from the bottom end portion thereof to the piezo-electric device housed at the top end portion thereof.

The conduit 2108 also includes a pair of flanges 2118 that are coupled to opposing outer side surfaces of the middle portion of the conduit 2108, and that extend laterally outward from the respective side surfaces as well as in a direction aligned with the overall length of the conduit 2108. The flanges 2118 each include a recess or cradle that is shaped and configured to cradle a portion of one of the batteries 2106, to partially restrain the batteries 2106 when the device 2100 is assembled. The internal cover 2112 includes a generally circular or disk-shaped main body portion and a hollow and truncated cone-shaped portion 2120 that extends upward from the main body portion. The main body portion of the internal cover 2112 includes a pair of openings or apertures 2122 that extend through the main body portion. Each of the apertures 2122 is sized and configured to cradle a portion of one of the batteries 2106, to partially restrain the batteries 2106 when the device 2100 is assembled. The external cover 2114 includes a generally circular or disk-shaped main body portion and an opening or aperture 2124 that extends through the main body portion. The aperture 2124 is sized and configured to fit snugly around a portion of the outer surface of the cone-shaped portion 2120 of the internal cover 2112 when the device 2100 is assembled.

FIGS. 6B, 6C, and 6D illustrate perspective, side, and cross-sectional side views, respectively, of the delivery device 2100. FIGS. 6E and 6F illustrate two different side views of the delivery device 2100 with the housing of the main body 2104 removed to reveal internal components of the main body 2104.

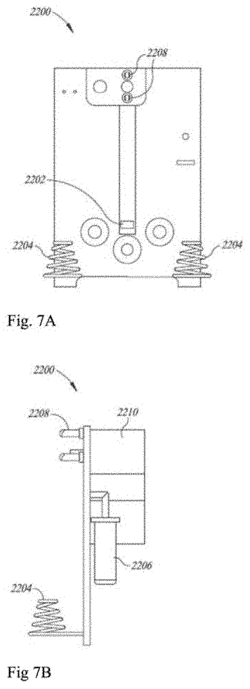

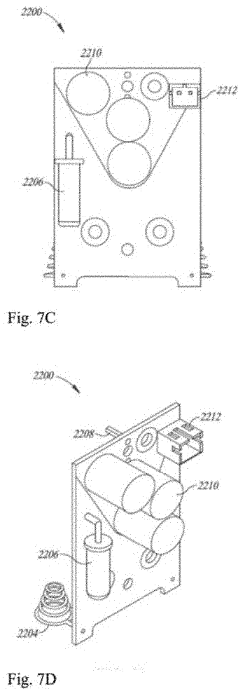

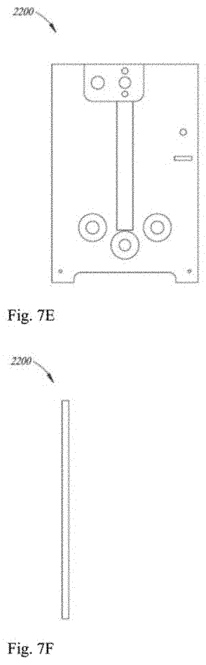

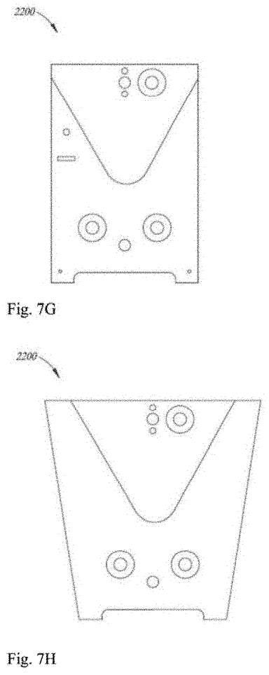

FIGS. 7A-7D illustrate the printed circuit board 2200 of the delivery device 2100 with associated components coupled thereto. FIG. 7A is a rear view of the printed circuit board 2200 and illustrates that the printed circuit board 2200 includes an LED 2202 physically and electrically coupled to the rear surface thereof, which can be operable to light up or turn on when the delivery device 2100 is generating a cloud of vaporized salt-based antimicrobial and/or anti-contagion formulation or composition or salt-based antimicrobial and/or anti-contagion formulation or composition in aerosol form, and to turn off when the delivery device 2100 is not generating a cloud of vaporized salt-based antimicrobial and/or anti-contagion formulation or composition or salt-based antimicrobial and/or anti-contagion formulation or composition in aerosol form. The LED can be useful to a user of the device 2100 because when the LED lights up, the user can be confident that power is being supplied to the printed circuit board 2200. FIG. 22A also illustrates that the rear surface of the printed circuit board 2200 is physically and electrically coupled to two metallic springs 2204, each of which is positioned and configured to act as a contact for, and to partially support or cradle, one of the batteries 2106. One of the springs 2204 can act as a positive contact, while the other of the springs 2204 can act as a negative contact, for the batteries 2106, such that the batteries 2016 will be installed within the device 2100 with their polarities reversed with respect to one another.

FIG. 7B is a side view of the printed circuit board 2200 and illustrates that the rear surface of the printed circuit board 2200 is also physically and electrically coupled to a plurality of gold pins 2208 to which a fluid sensor can be physically and electrically coupled. FIG. 7C is a front view of the printed circuit board 2200 and illustrates that the front surface of the printed circuit board 2200 can include an electrical connector 2212, which can be a JST connector, to allow an operator to physically and electrically couple other electronic devices, such as the piezo-electric device 2110, to the printed circuit board 2200 and to allow the printed circuit board and other associated components coupled thereto to communicate with (e.g., transmit signals to or receive signals from) such other electronic devices including the piezo-electric device 2110. FIG. 7C also illustrates that the front surface of the printed circuit board 2200 is physically and electrically coupled to a tilt sensor 2206, which can include an accelerometer or a ball tilt switch in which a ball moves and connects pins to complete an electrical circuit when the device 2100 is tilted, and to a plurality of capacitors 2210 for storing electrical energy. FIG. 7D is a perspective view of the printed circuit board 2200 and illustrates a perspective view of the printed circuit board 2200 with the associated components coupled thereto. FIGS. 7E-7G illustrate the printed circuit board 2200 without the associated components coupled thereto.

As illustrated in FIGS. 6A-6F, the rear of the printed circuit board 2200, illustrated directly in FIG. 7A, faces toward the conduit 2108 and the center of the delivery device 2100, while the front of the printed circuit board 2200, illustrated directly in FIG. 7C, faces away from the conduit 2108 and the center of the delivery device 2100. In some implementations, the printed circuit board 2200 receives power from a source at between 2.0 and 3.4 Volts DC, and provides power to a load at 140 KHz and at 65 Volts peak-to-peak. FIG. 7H illustrates a front view of an alternative shape and configuration for the printed circuit board 2200. FIGS. 7A-7H illustrate some examples of possible dimensions of the printed circuit board 22, with the numbers used in millimeters. It will be understood that the specific dimensions provided in these Figures are merely examples of possible suitable dimensions.

To operate the delivery device 2100, a user can fill the base 2102 with salt-based antimicrobial and/or anti-contagion formulation or composition in a liquid form and assemble the device 2100 except for the batteries 2106 and the external cover 2114, such as by screwing or threading the base 2102 onto the main body 2104. The user can then insert the batteries 2106 into the device 2100 through the apertures 2122 in the internal cover 2112, such that the batteries are partially cradled by the recesses of the flanges 2118, and such that bottom terminals of the batteries 2106 are in electrical contact with the springs 2204. The user can then couple the external cover 2114 to the rest of the device 2100, such as by threading or press-fitting the external cover into a top end of the main body 2104. An underside of the external cover 2114 can include a strip of electrically-conductive material, such as metal, which can engage the top terminals of the batteries 2106 and electrically couple the upper terminal of one of the batteries 2106 to the upper terminal of the other one of the batteries 2106.

The user can then lift and tilt the device 2100, such that the fluid flows, under the force of gravity, from the base 2102, through the conduit 2108, to the piezo-electric device 2110. Once the user tilts the device 2100, for example to dock with the primary vessel, the tilt sensor 2206 can generate and transmit a signal indicating that the device 2100 has been tilted. Further, once the fluid flows to the piezo-electric device 2110, the fluid may come into contact with a fluid sensor coupled to the pins 2208 and generate and transmit a signal indicating that the fluid has reached the fluid sensor. Further still, the device 2100 can include a pressure-sensitive switch on a bottom surface thereof which, when the device 2100 is picked up off of a flat surface, can generate and transmit a signal that the device 2100 has been picked up. In some implementations, the device 2100 includes no manually-operated switches or buttons, and receives no input from the user, other than one, two, or three of the signals described above.

Upon receipt of any one, any two, or all three of such signals, the device 2100 can activate the piezo-electric device 2110 to begin generating a cloud of vaporized salt-based antimicrobial and/or anti-contagion formulation or composition or salt-based antimicrobial and/or anti-contagion formulation or composition in aerosol form from the scent media in liquid form. Because the device 2100 is tilted sideways or upside-down, the cloud of vaporized salt-based antimicrobial and/or anti-contagion formulation or composition or salt-based antimicrobial and/or anti-contagion formulation or composition in aerosol form can flow out of the device 2100 through the hollow cone-shaped portion 2120, and can be dispensed into free space to be consumed directly by the user or can be poured into another container or vessel for subsequent consumption by the user. In some implementations, the device 2100 includes an internal timer and automatically turns off or de-activates the piezo-electric device 2110 to stop generating the cloud of vaporized salt-based antimicrobial and/or anti-contagion formulation or composition or salt-based antimicrobial and/or anti-contagion formulation or composition in aerosol form after a time period of about 5, about 10, about 15, or about 20 seconds. In other implementations, the device 2100 continues to operate and generate the vaporized salt-based antimicrobial and/or anti-contagion formulation or composition or salt-based antimicrobial and/or anti-contagion formulation or composition in aerosol form until the device 2100 is once again oriented upright or placed back on a flat horizontal surface.

When the fluid within the base 2102 runs out, the user can unscrew or unthread of the base 2102 from the main body 2104 of the device 2100, refill the base 2102 with more of a salt-based antimicrobial and/or anti-contagion formulation or composition in a fluid form, screw or thread the base 2102 back on to the main body 2104, and then resume using the device 100. When the batteries 2106 die, no longer power the device 2100, and need to be replaced, the user can remove the external cover 2114 from the rest of the device 2100, such as by unscrewing, unthreading, or turning the external cover 2114 with respect to the rest of the device 2100. The old batteries 2106 within the device 100 can then be removed and new batteries 2106 can be installed in their place. The user can then re-install the external cover 2114 onto the rest of the device 2100 and resume using the device 2100.

In some implementations, the external cover 2114, or a surface of the rest of the device 2100 that engages with the external cover 2114, includes a detent, and the detent is engaged as the external cover 2114 is turned with respect to the rest of the device 2100 just before the external cover 2114 is released from the rest of the device 2100. Engagement of the detent can serve as a signal to the user that the external cover 2114 is about to be released from the rest of the device 2100. Once the user releases and removes the external cover 2114 from the rest of the device 2100, the batteries are disconnected and the device is unable to operate. Thus, the external cover 2114 can act as a switch, where removing the external cover 2114 from the rest of the device 2100 switches the device 2100 off and engagement of the external cover 2114 with the rest of the device 2100 switches the device 2100 into an ON state.

### EXPERIMENTAL

It was recently observed that the delivery of a nasal saline comprised of a mist of 10 μm droplets containing a mixture of calcium and sodium chloride salts can reduce respiratory droplets by up to 99% for up to 6 hours post administration. The salts (a hypertonic mixture called Composition A) associate with mucin macromolecules near the mucus surface, binding mucus molecules together, thereby increasing mucus surface tension and surface viscoelasticity. These effects help mucus surfaces withstand the stresses that occur on air passing over mucus during normal breathing, resulting in fewer respiratory droplets in the airways, and fewer exhaled aerosol particles—a form of “airway hygiene.”

Airway hygiene follows a millennia-long tradition of nasal saline administration for cleaning mucus surfaces of foreign particulate matter. Salts ranging from pure sodium chloride (table salt) at physiological tonicity (0.9% by weight) to more complex mixtures of salts including calcium chloride, magnesium chloride and others, have long been commonly administered as gavages and nasal sprays. Hypertonic salt compositions can particularly increase cilia beat, facilitating the clearance of mucus and associated particulate matter toward the mouth.

The effects of airway hygiene were explored in learning establishments in the USA (Grand Rapids Mich. and Cape Cod Mass.) and in comparison with a setting (Bangalore India) of relatively high airborne particulate burden. Two kinds of nasal saline administration were examined—Composition A and Simply Saline Nasal Spray. The findings are reported below.

### Methods

Human volunteer studies were conducted at Bangalore Baptist Hospital (BBH), Grand Rapids Community College (GRCC), and Cape Cod Academy (CCA). IRB approval was received for these (non-drug cosmetic) studies from the BBH Ethics Committee, the GRCC Ethics Committee, and for the CCA study a waiver of the need for IRB approval from E & I Review, an independent accredited ethics review board. Forty 40 human volunteers were recruited in Bangalore, ages 26 to 63; 120 human volunteers in Grand Rapids, ages 11 to 68; and 93 human volunteers on Cape Cod, ages 10 to 70. Overall 126 females, 104 males, and 23 undeclared persons were recruited. Of the 253 human subjects, 82 were Caucasian, 15 were African American, 33 were Asian, 33 were Latino, 49 were Indian, and 41 were undeclared. Participants were not screened for SARS CoV-2 infection by serology or polymerase chain reaction (PCR) before enrollment. While none of the subjects were known to be COVID-19 positive, two of the subjects in Grand Rapids, a brother (16 years of age) and a sister (19 years of age) living in the same household, were suspected to be asymptomatic carriers given an unusually high exhaled aerosol number and sibling relationship. All participants in all studies provided written informed consent prior to enrollment. FIG. 8 illustrates the general design of the protocol for the three study sites (specifically the GRCC study site).

Exhaled particles were measured, before and after nasal saline administration, by a particle detector (Climet 450-t) designed to count airborne particles in the size range of 0.3 to 5 micrometers. The particle detector was connected to standard nebulizer tubing and mouthpiece that filters incoming air through a HEPA filter. Each standard nebulizer tubing and mouthpiece was removed from sealed packaging before each subject prior to the subject's first exhaled particle detection. On subsequent counting maneuvers the same mouthpiece, tubing and HEPA filter were replaced into the particle counter system by the participant to insure effective hygiene. Subjects performed normal tidal breathing through a mouthpiece while plugging their noses over 1 to 2 minutes—beginning with two deep breaths to empty their lungs of environmental particles. Over this time frame particle counts per liter diminished to a lower baseline number reflecting particles emitted from breakup of airway lining fluid surfaces in the subject's airways. Once the lower plateau of particle counts was reached subjects continued to breathe normally. Three to eight particle counts (average values of particle counts assessed over six seconds) were then averaged to determine the mean exhaled particle count and standard deviation, Participants sat opposite to the study administrator with a plexiglass barrier in between.

Two nasal salines were used in the studies. Composition A is a drug-free nasal saline hygiene formulation comprised of calcium chloride and sodium chloride in distilled water. Overall salt composition (4×isotonic composition) is in the range of sea water, specifically with 0.43M CaCl₂, 0.05M NaCl (4.72% CaCl₂, 0.31% NaCl). Composition A compositions were manufactured at Pharmasol (MA) in a GMP mixing and filling facility and contained in sealed plastic bottles (0.5 ounces). Composition A bottles were opened and emptied into glass vials of the Mister device. The hand-held, vibrating-mesh nebulizer is produced at Perfect Electronics in Shenzhen, China, with a 6 μm pore size to produce, on tipping of the device, an aerosol cloud with a particle size distribution optimal for delivery to the nose through natural nasal inspiration. Generating a median volume particle diameter of 9-10 μm (16), optimal for nasal and upper airway deposition of aerosol following a deep natural tidal inspiration through the nose and with relatively uniform distribution of deposition from the anterior to the posterior of the nose, Nimbus produces on tipping 57 mg+/−2 mg within a 10 second actuation, after which power ceases until tipped back upright and again overturned. The device delivers a dose of approximately 33 mg (1.56 mg CalCl₂) by filling an empty 6 oz glass with the cloud for the internally programmed 10 s actuation of the device and then inspiring the cloud directly from the glass into the nose. Dosing can also be achieved by creating the cloud before the nose with deep nasal inspiration.

Simply Saline by Arm & Hammer, a nasal spray of isotonic sodium chloride available on the market, was bought and used (one spray per nostril) as a control.

### Results

### Total Exhaled Aerosol Versus Ambient Inhaled Particle Mass Exposure

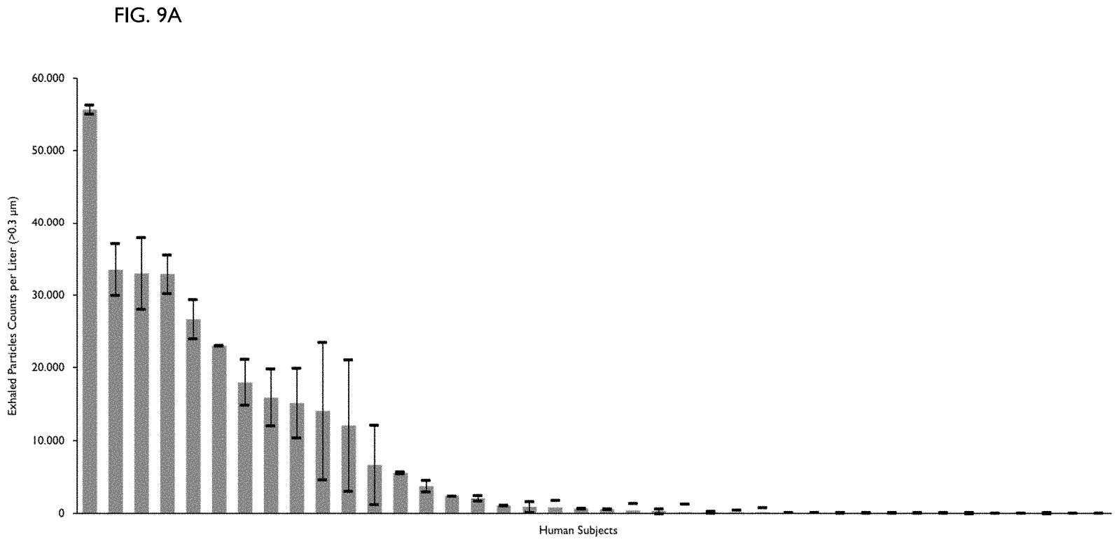

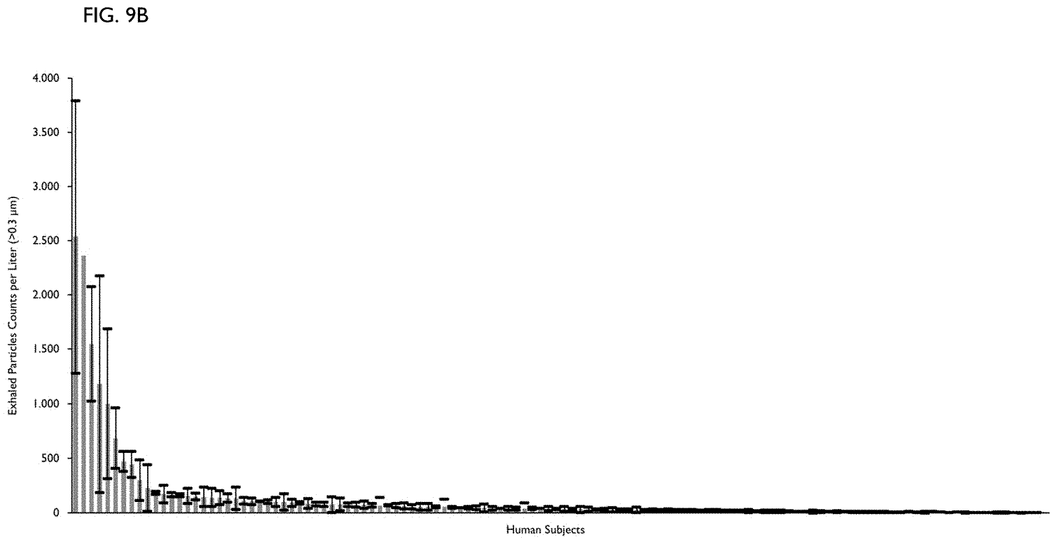

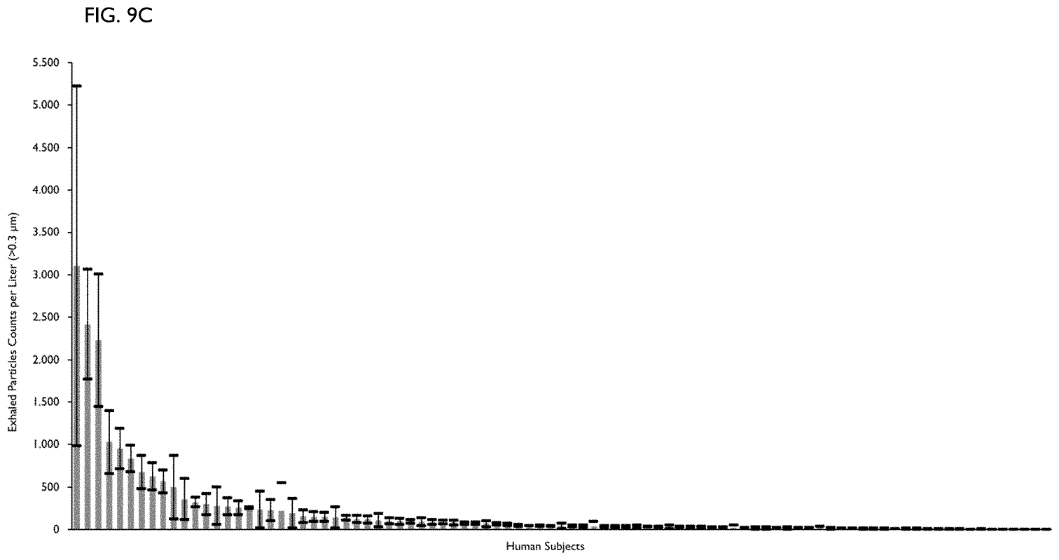

Exhaled aerosol particle numbers and sizes at the three sites were assessed, including 40 human subject volunteers in Bangalore (FIG. 9A), 120 human volunteers in Grand Rapids (FIG. 9B), and 93 human volunteers on Cape Cod (FIG. 9C). At each site a small group of subjects exhaled around 80% of the overall aerosol of the group, adhering to the classical 20:80 rule of “super spreading” of infectious disease.

“Super Spreaders” (of aerosol particles) are considered those individuals who exhale 80% of total aerosol particles of the group (while being less than 20% of all subjects). Classic super spreader distributions can be found at each US sites: in Grand Rapids 24 (20%) of the 120 subjects produce 79.5% of the exhaled aerosol of the group, while at the Cape Cod site 19 (20%) of the 93 subjects produce 79.7% of the exhaled aerosol of the group. However, only 10% (4 of the 40) subjects in India produce 82.6% of the exhaled aerosol of the group, while 20% of all subjects (8 subjects) produce 95.1% of all exhaled aerosol.

The skewing of the importance of high emitters in India reflects the fact that exhaled aerosol numbers are dramatically higher at the Indian relative to the US sites. The top 20% aerosol emitting individuals in Bangalore had mean exhaled aerosol particle numbers of 30,585+/−11,380. Mean exhaled aerosols were statistically lower (p<0.0002) at the US sites, notably 532+/−668 in Grand Rapids and 818+/−722 on Cape Cod. These differences mirror the differences in burden of airborne particulate matter between the Indian and US sites. Reported particulate mass (PM10) smaller than 10 μm over this same time frame in India (Bangalore) was 150 μg/m3 while for the USA sites it was an order of magnitude lower (17 μg/m3 in Grand Rapids and 7 μg/m3 on Cape Cod).

No statistically significant differences were observed in our study across sites between exhaled aerosols among Caucasian, African American, Asian, Hispanic or Indian subjects, nor were significant differences observed between subjects of varying age or BMI. Correlations between exhaled aerosol and BMI years (BMI multiplied by age) was observed, while differences in BMI years between the three study sites were not significant.

### Exhaled Aerosol Versus Time Post Airway Hygiene Administration

The effect of airway hygiene on exhaled aerosol in Bangalore was evaluated as a function of time post administration and in comparison to the saline nasal spray control.

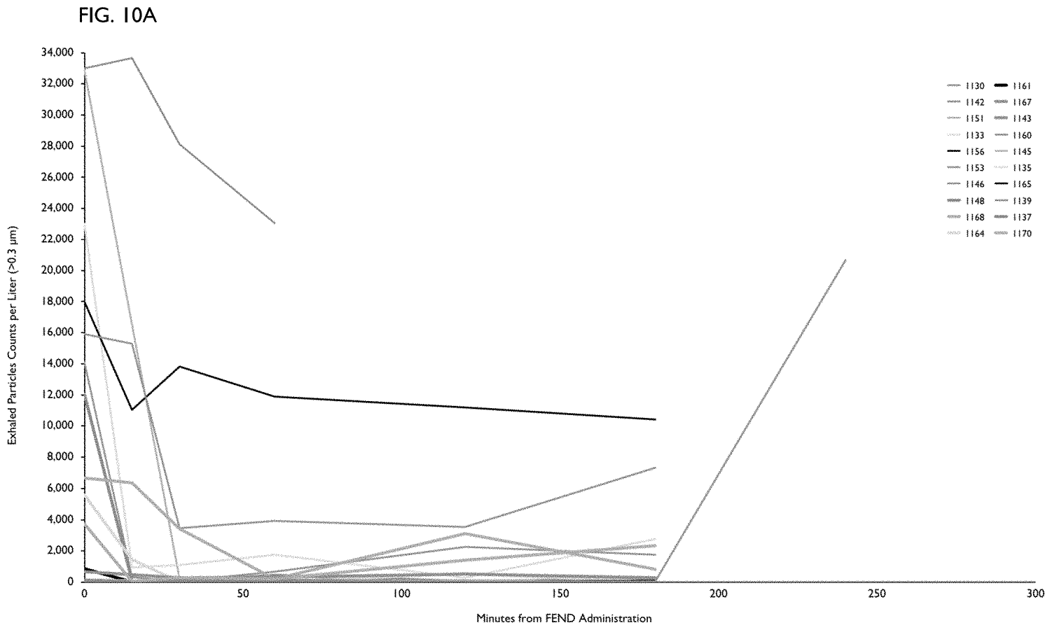

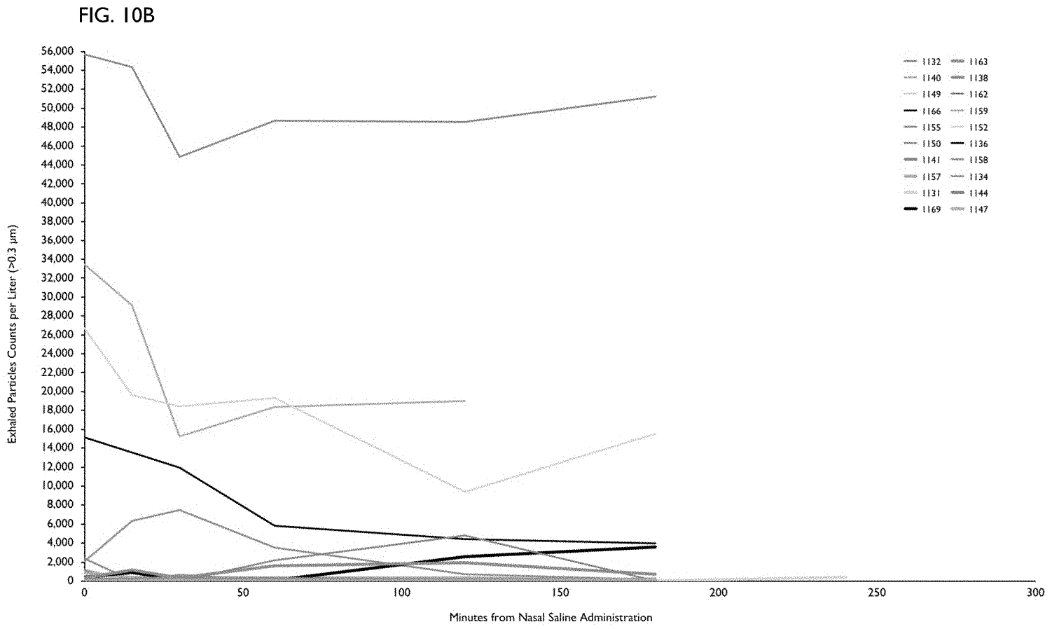

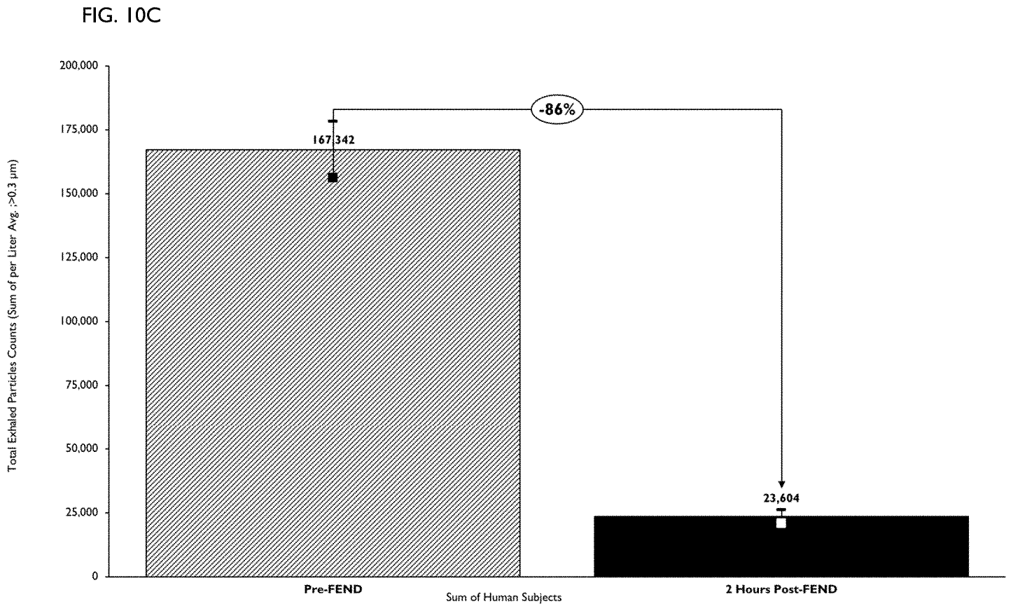

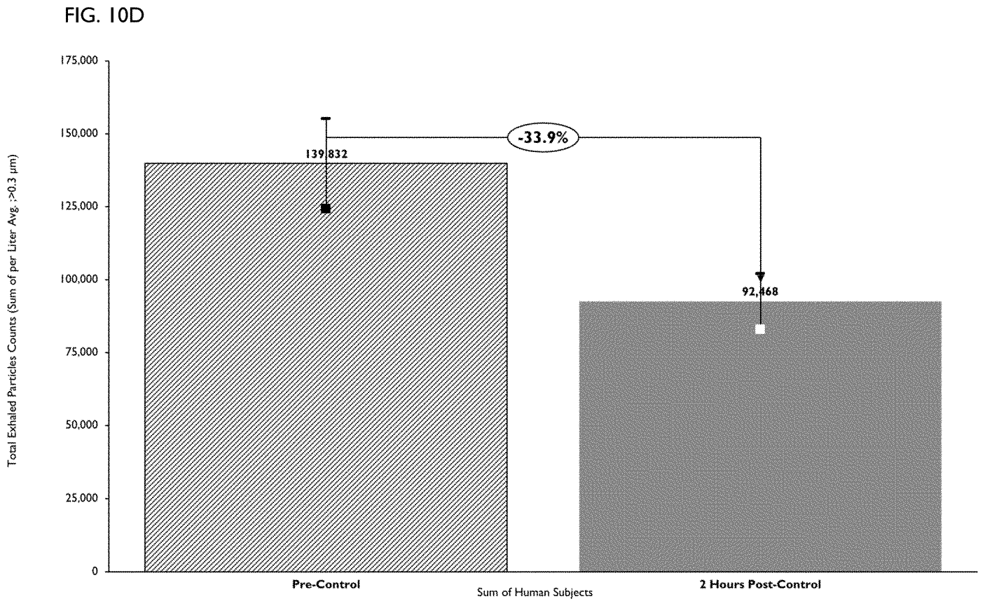

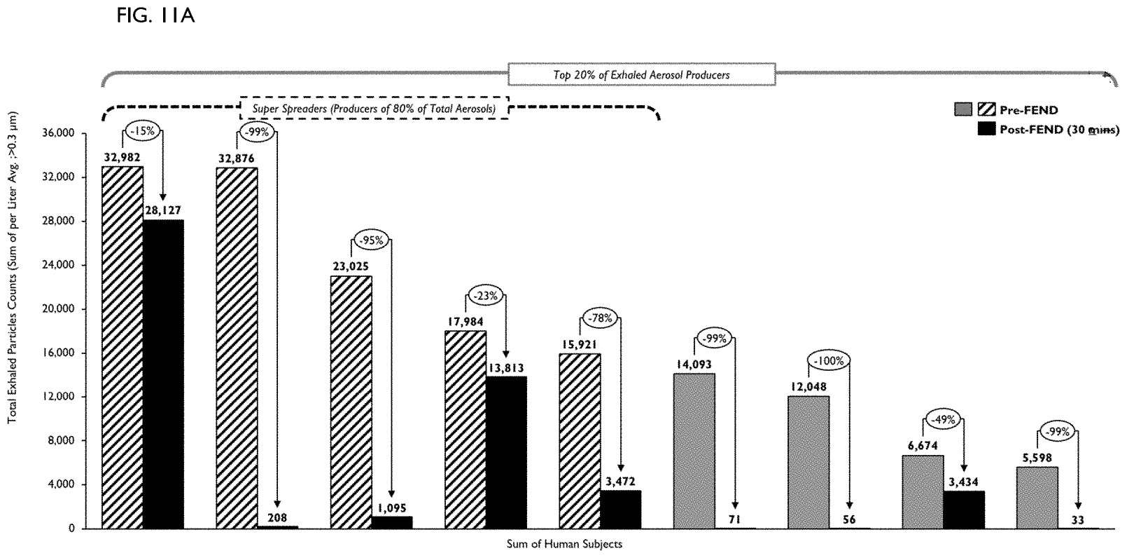

In the case of those subjects who received Composition A (n=20), post administration, exhaled aerosol numbers fell within 15 minutes and remained suppressed for at least three to four hours (FIG. 10A). In the case of those subjects who received the saline nasal spray control (n=20), post administration exhaled aerosol numbers fell to a lesser degree, and were mixed over the several hours post administration (FIG. 10B).

FIGS. 10C and 10D present the suppression effect following Composition A versus the nasal saline control on overall exhaled aerosol of all 40 subjects at two hours post dosing. The large difference in overall exhaled aerosol relative to baseline (86%) was highly significant (p<0.011) for the Composition A airway hygiene (n=20), while the diminution in overall exhaled aerosol (34%) was insignificant (p<0.62) for the Simply Saline control (n=20).

### Exhaled Aerosol Suppression by Airway Hygiene Administration

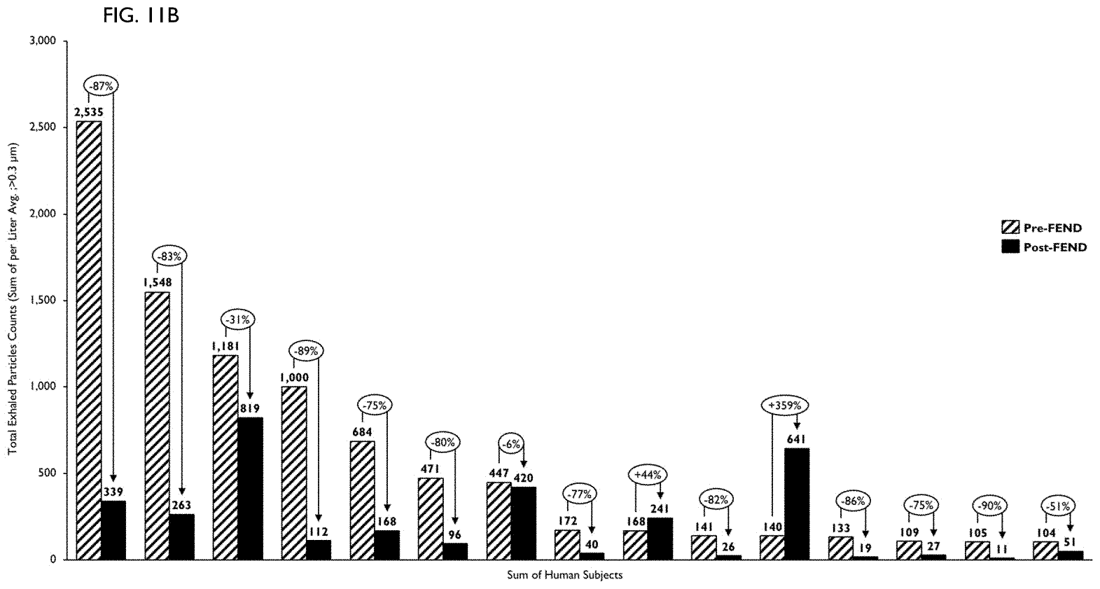

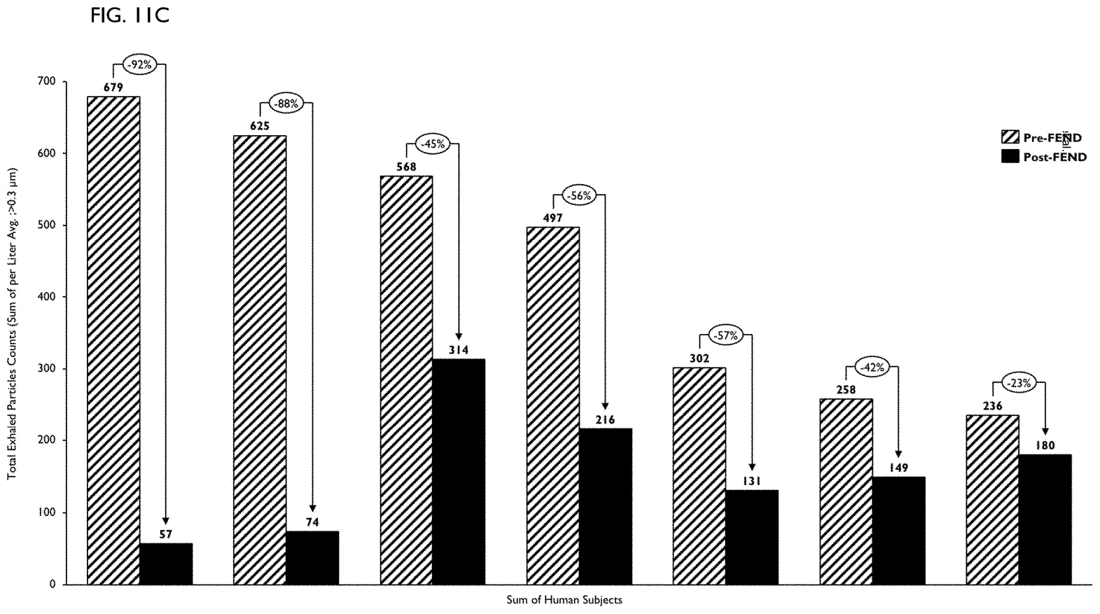

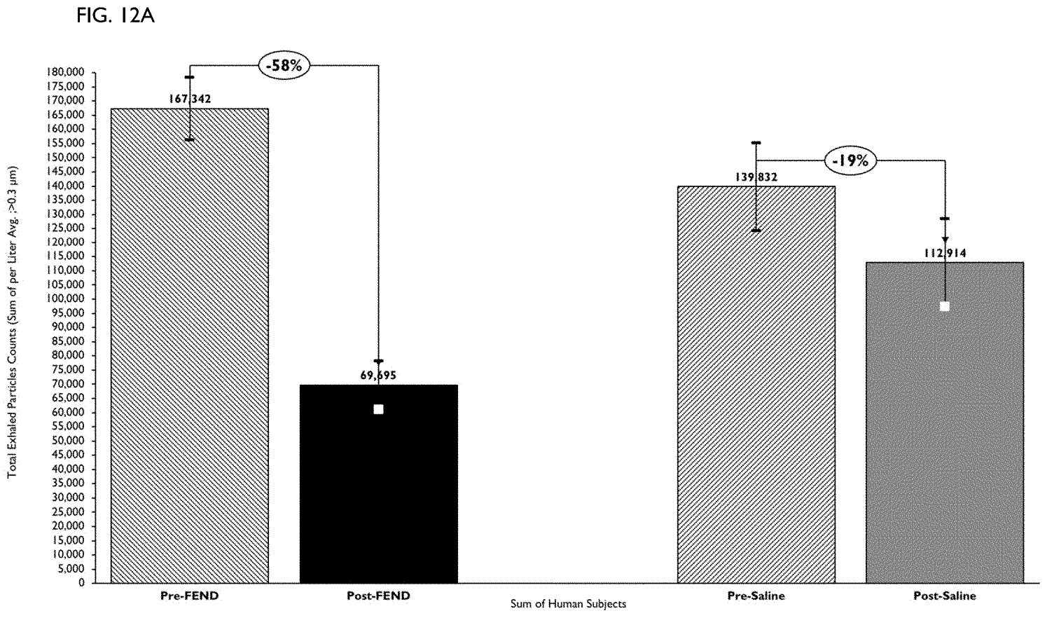

The effectiveness of nasal saline airway hygiene was evaluated in Bangalore, Grand Rapids and Cape Cod by evaluating exhaled aerosol from all subjects before and 15 to 30 minutes post administration of Composition A or Simply Saline. The results for the 20% highest emitting aerosol subjects are shown in FIGS. 11A-C(Composition A).

Composition A administration reduces exhaled aerosol most significantly in the airways of those exhaling the greatest numbers of aerosol particles at each site, with the most significant % reductions appearing in the dirtiest air environment, notably Bangalore, where exhaled aerosol is most significantly elevated. The less significant individual subject Composition A suppression relative to baseline seen in FIG. 11A relative to FIG. 10C relates to the continued decline in exhaled aerosol with time post Composition A administration (FIG. 10A).

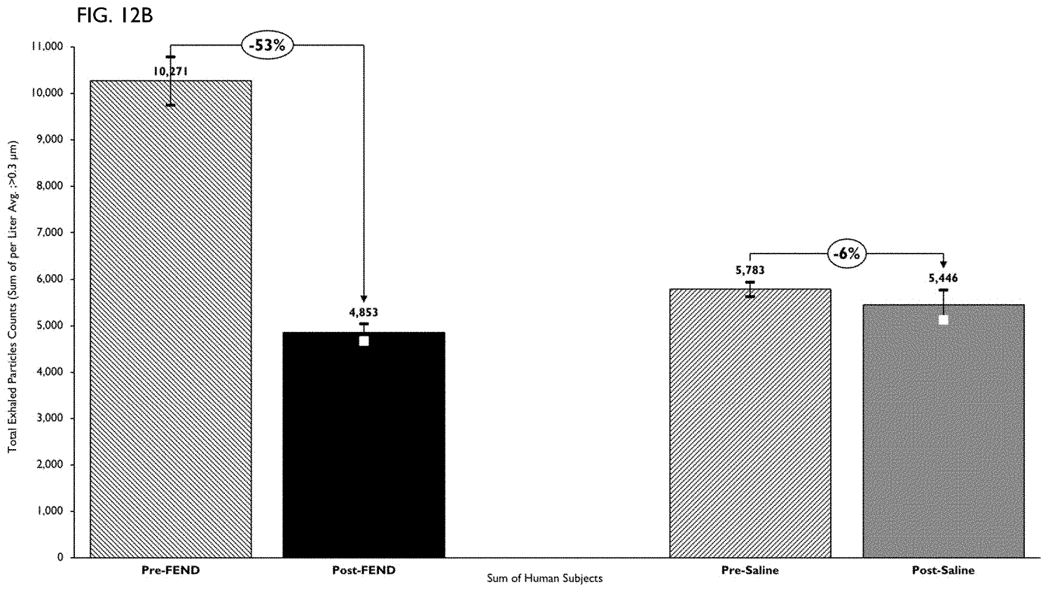

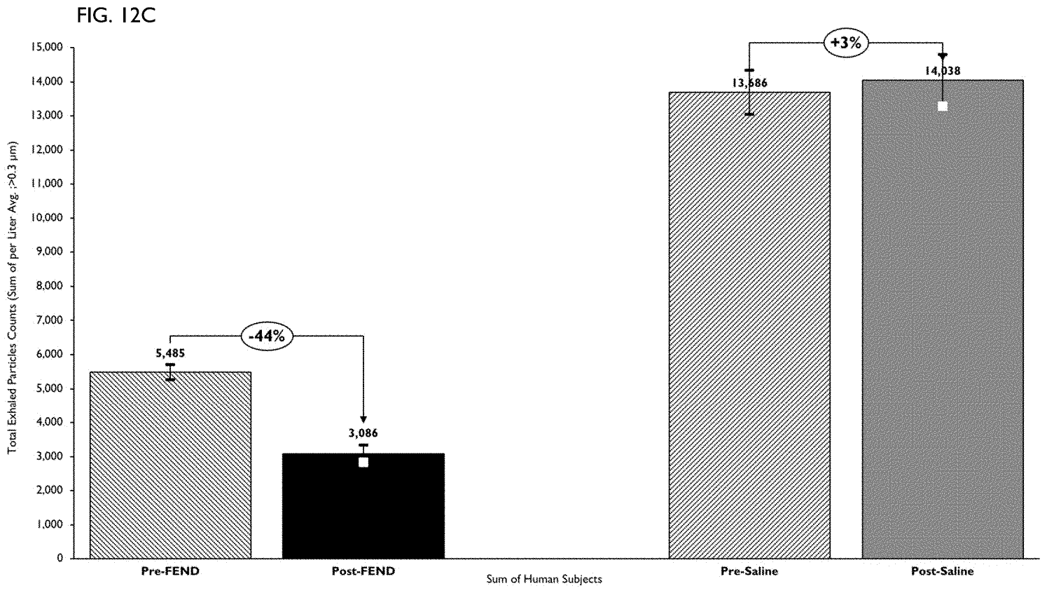

FIGS. 12A-C present the overall degree of suppression of exhaled aerosol at each site for both Composition A and Simply Saline at 15 to 20 minutes post administration. Overall airway cleansing by the Simply Saline control is insignificant in every case (BBH p<0.94, GRCC p<0.83, CCA p<0.65), while the overall Composition A airway cleansing effect is marginally significant at each site of the study (BBH p<0.169, GRCC p<0.124, CCA p<0.098), reflecting the large dispersion in exhaled aerosol numbers between Low Spreaders and Super Spreaders (FIGS. 11A-C).

### Discussion

The study of exhaled aerosol in India and the USA suggests that prolonged inhalation of high levels of micron- and submicron particulate matter may promote the generation of large numbers of respiratory droplets, and skew these droplets to submicron size (FIG. 9). These findings are consistent with the hypothesis that inhaled particles, by landing on mucus surfaces, lower surface tension and surface viscoelasticity, rendering airway lining mucus more prone to breakup into droplets of smaller size.

These same trends were observed elsewhere in exhaled aerosol following viral (COVID-19) and bacterial (tuberculosis) infection in nonhuman primates. Exhaled aerosol increases, and exhaled aerosol particle size decreases, in tandem with proliferation of viral and bacterial burden in lung tissues.

Whether or not, in the development of respiratory diseases such as COVID-19 and tuberculosis, the accumulation of viral and bacterial particles at or near mucus surfaces has a similar surface property alteration effect as the accumulation of fine particles breathed in from the atmosphere—the findings of this study suggest that an abundance of respiratory droplets play a role in the spread of airborne infectious disease in dirty air settings. This might also explain the trends that have been observed for heightened risk of COVID-19 death in polluted settings.

Airway hygiene is a simple hygienic intervention that can allow people to meet the respiratory droplet carrier challenge wherever they live. Administration of the calcium-enriched nasal saline, by increasing surface viscoelasticity via calcium-mucin interactions, suppresses droplet breakup, cleaning the upper airways of respiratory droplets by up to 99% in the dirtiest airways (FIG. 9C) for several hours (FIG. 9A). The effectiveness of airway hygiene obviously can be influenced on the success of the application (i.e. deep nasal inspiration) technique, while it appears significant within 15 minutes of administration (FIG. 12) and is remarkably consistent across all the sites of and all subjects. It is especially effective for those breathing dirty air (FIGS. 10A, 10C).

By comparison the nasal saline spray control has little to modest effect—no overall effect was observed in the short-term (15-30 minute) time frame of FIGS. 12A-C while an indication of effect in FIG. 10B over time. This conclusion is consistent with the belief the inhalation of isotonic saline suppresses exhaled aerosol, and may suggest that post-nasal drip drainage of the saline solution from the nose can reach the trachea in some subjects.

It has been found that children and young adults (under the age of 26) exhale very few particles. In the 120 children and young adults assessed, only 6 of these young people were observed to exhale more than 150 particles per liter, while 89% of the young subjects exhaled between 1 and 50 particles per liter of air. Of these six individuals all of these exceptions exhaled aerosols exceeding 1000 particles per liter. In three of these cases, subjects 5, 8 and 39 from our Bangalore study, the young subjects breathed into their airways highly polluted air. In two of the cases, the Grand Rapids siblings (16 and 19 years of age), the children were suspected to have been asymptomatic carriers of COVID-19, while in one case, a 17 year-old male on Cape Cod, there was no obvious airway particle burden causality. It appears that while young people as a rule have very few respiratory droplets, they can become high producers of respiratory aerosols, and especially should their airways be overridden by foreign particles, whether inbound particulate matter, or—as in the case of infection—proliferating virus.

While a combination of environmental and biological factors clearly renders certain people more vulnerable than others to respiratory disease, and more susceptible than others to the communication of airborne infectious disease, including COVID-19, the results suggest that by weakening airway lining mucus, inhaled particles may themselves be at the origin of excessive respiratory droplet creation, and risk of infection and transmission.

### EXAMPLES

Example 1. A method of administering a formulation or composition or a therapeutic formulation or composition to the nose, trachea, and main bronchi of a respiratory tract of a subject, method comprising:

generating an aerosol of droplets in a space from which the aerosol is naturally inspirable by the subject, in the nose, trachea, and main bronchi of the respiratory tract of the subject, without any application of force; wherein the aerosol of droplets comprises a salt-based therapeutic composition comprising calcium chloride and wherein in water, the droplets have a mass median droplet diameter ranging from approximate 7 microns to approximately 15 microns

Example 2. The method of example 1 wherein the droplets comprise greater than 1% by weight of calcium chloride.

Example 3. The method of example 1 wherein the salt-based composition comprises both calcium chloride and sodium chloride.

Example 4. The method of example 1 wherein the salt-based composition further comprises an essential oil, fragrance oil or flavor extract selected from the group consisting of cacao oil, caramel oil, cinnamon bark oil, coffee oil, eucalyptus oil, palm oil, fig oil, grapefruit oil, hazelnut oil, honeydew melon oil, lavender or spike lavender oil, lemongrass oil, lime oil, black or green pepper oil, peppermint oil, rosemary oil, strawberry oil, smoke oil, tobacco vanilla oil, vanilla oil, chocolate extract, anise extract, rose linalool, and combinations thereof.

Example 5. The method of example 1 wherein the salt-based composition comprises calcium chloride and 10% by weight ethyl alcohol.

Example 6. The method of example wherein the droplets comprise generating greater than 4% by weight calcium chloride.

Example 7. The method of any of examples 1 through 6 wherein the droplets have a mass median droplet diameter ranging from 9 microns to 10 microns.

Example 8. The method of any of examples 1 through 6 wherein the droplets have a mass median droplet diameter of approximately 10 microns, with a standard deviation of less than 1 micron.

Example 9. The method of any of examples 1 through 6 wherein the droplets have a mass median droplet diameter ranging from 7 microns to 15 microns, with a standard deviation of less than 1 micron.

Example 10. The method of any of examples 1 through 6 wherein a majority of the droplets have a droplet size between 9 microns and 10 microns in diameter.

Example 11. The method of claim 10 wherein a majority of the droplets have a droplet size of approximately 10 microns in diameter.

Example 12. The method of any of examples 1 through 6 wherein each of the droplets comprise between 0.5-4.0 mg calcium chloride.

Example 13. The method of any of examples 1 through 6 wherein the generating step comprises providing the aerosol into a free space.

Example 14. The method of claim 13 wherein a velocity of the aerosol is slowed down relative to a velocity of the aerosol as it leaves a dispenser and from which the aerosol becomes relatively quiescent.

Example 15. The method of any of examples 1 through 6 wherein the aerosol is provided in a range of 12 inches to 1 inch of a nose of the subject.

Example 16. The method of any of examples 1 through 6 wherein the aerosol is provided in a range sufficient distant to a nose of the subject such that the aerosol has zero or negligible net velocity at least horizontally with respect to the earth.

Example 17. The method of any of examples 1 through 6 wherein the generating step includes providing the aerosol in an at least partially constrained space in the form of a vessel from which the aerosol is inspirable via an opening in the vessel.

Example 18. The method of any of examples 1 through 6 wherein the generating step comprises the aerosol for a defined period of time in response to an activation event, and ceasing the generating after the defined period of time until a subsequent activation event.

Example 19. The method of any of examples 1 through 6 wherein the generating step comprises repeatedly generating the aerosol for defined periods of time, the defined periods of time separated by periods of time during which the generating of the aerosol ceases, to deliver multiple doses over a period of time.

Example 20. The method of any of examples 1 through 6 wherein the generating step comprises providing the aerosol in a free space in a venue prior to and/or during an event.

Example 21. The method of claim 20 wherein the aerosol is provided in a free space at an entrance to the venue.

Example 22. The method of any of examples 20 or 21 wherein the aerosol is provided in a free space at a queue for the event, through which subjects successively pass and the providing occurs continuously or periodically over an extended period of time during which access to the event is provided.

Example 23. The method of claim 22 wherein the aerosol is provided at two or more locations along a length of a queue path used to access the event.

Example 24. The method of claim 22 wherein the aerosol is provided along an entire length of at least a defined portion of a queue path used to access the event, wherein the defined portion is sufficiently long to provide a measured dosage to each subject traversing the defined portion of the queue path at a walking speed.

Example 25. The method of any of examples 22 through 24, further comprising:
successively reading identification information from each subject passing through the aerosol; and
storing the information that represents that each subject passed through the aerosol.

Example 26. The method of example 24, further comprising:

successively reading identification information from each subject passing through the aerosol via at least one machine-readable symbol reader, radio frequency identification (RFID) interrogator, or via facial recognition based camera and processor-based computer system; and

storing the information to at least one non-transitory processor-readable media that represents an amount of time that each subject was subjected to the aerosol.

Example 27. The method of any of examples 20 through 26 wherein the aerosol droplets have a mass median droplet diameter of approximately 10 microns, with standard deviation of less than 1 micron.

Example 28. The method of any of examples 20 through 26 wherein a majority of the droplets have a droplet size ranging from 7 microns to 15 microns in diameter.

Example 29. The method any of examples 20 through 26 wherein each of the droplets comprise between 0.5-4.0 mg calcium chloride.

Example 30. The method any of examples 20 through 26 wherein the salt-based composition is a purely hygienic composition.

Example 31. The method any of examples 20 through 26 wherein the subject is administered a therapeutically effective amount of the salt-based composition.

Example 32. A method of suppressing the exhalation of particles in an upper airway of a respiratory tract of a subject, the method comprising the generating an aerosol of droplets, and administering the aerosol of droplets to the airway lining fluid in the nose, trachea, and main bronchi of the subjects, thereby suppressing the exhalation of particles in the upper respiratory tract of the subject, wherein:

the aerosol of droplets comprise a salt-based composition comprising at least calcium chloride in water droplets, the droplets have a mass median droplet diameter ranging from approximately 7 microns to approximately 15 microns, and the droplets are suspended in a standing cloud.

Example 33. The method of example 32 wherein the composition consists of water and calcium chloride.

Example 34. The method of example 32 wherein the composition further comprises sodium chloride.

Example 35. The method of any of examples 32 through 34 wherein the droplets have a mass median droplet diameter of between 9 and 10 microns, and a standard deviation of less than 1 micron.

Example 36. The method of example 32, wherein composition further comprises:

an essential oil, fragrance oil or flavor extract selected from the group consisting of cacao oil, caramel oil, cinnamon bark oil, coffee oil, eucalyptus oil, palm oil, fig oil, grapefruit oil, hazelnut oil, honeydew melon oil, lavender or spike lavender oil, lemongrass oil, lime oil, black or green pepper oil, peppermint oil, rosemary oil, strawberry oil, smoke oil, tobacco vanilla oil, vanilla oil, chocolate extract, anise extract, rose linalool, and combinations thereof.

Example 37. The method of example 36, wherein the composition further comprises:

linalool-containing essential oils.

Example 38. The method of example 32, wherein the composition further comprises:

ethanol at a concentration greater than 5% by weight.

Example 39. The method of example 34 wherein the calcium chloride and sodium chloride are dissolved in water and the resulting solution is formable into an aerosol of droplets having a median droplet diameter of between 9 microns and 10 microns.

Example 40. The method of example 34 wherein the calcium chloride and sodium chloride are dissolved in water and the resulting solution is formable into an aerosol of droplets having a mass median droplet diameter of approximately 10 microns, with standard deviation of less than 1 micron.

Example 41. The method of example 32 wherein the calcium chloride is administered to the airways in an aerosolized form, wherein each droplet contains between 0.5-4.0 mg calcium chloride.

Example 42. The method of any of examples 32 through 41 wherein the composition is formable via a nebulizer into an aerosol of droplets having a mass median droplet diameter of approximately 10 microns, with standard deviation of less than 1 micron.

Example 43. A delivery system operable to delivery of a purely hygienic or a therapeutic antimicrobial formulation or composition to the nose, trachea and main bronchi of a respiratory tract of a subject, the delivery system comprising:

a reservoir having at least one wall which at least partially delimits an interior of the reservoir from an exterior thereof, the reservoir having a port that provides a fluidly communicative path between the interior of the reservoir and an exterior thereof, the reservoir which at least in use holds the hygienic or therapeutic antimicrobial formulation or composition comprising a quantity of water and at least calcium chloride dissolved in the water;

at least one nebulizer delivery device, the at least one nebulizer delivery device comprising a reservoir and an actuator, and the actuator controllably operable on the active substance media to cause formation of an aerosol comprising readily-soluble droplets that have a mass median diameter range of approximately 7 microns to approximately 15 microns and comprising at least the calcium chloride dissolved in the quantity of water.

Example 44. The delivery system of example 43 wherein the at least one nebulizer delivery device is a nebulizer and further comprises a respective control subsystem communicatively coupled to control the actuator.

Example 45. The delivery system of example 43 wherein the at least one nebulizer delivery device is a nebulizer that includes a mesh screen mounted for oscillation, a microcontroller, and at least one of a piezoelectric transducer, a solenoid, or an electric motor drivingly coupled to oscillate the mesh screen along at least one axis in response to signals from the microcontroller to dispense aerosol.

Example 46. The delivery system of example 45, further comprising: at least one of a switch or a sensor communicatively coupled to the microcontroller and operable to produce a signal that causes the microcontroller to operate the actuator accordingly.

Example 47. The delivery system of example 45, further comprising:

at least one of a switch or a sensor communicatively coupled to the microcontroller and operable to produce a signal that causes the microcontroller to operate the actuator in response to the at least one nebulizer delivery device being titled relative to a normal or upright position.

Example 48. The delivery system of example 45, further comprising:

at least one of a switch or a sensor communicatively coupled to the microcontroller and responsive to a position or orientation of the vessel and operable to produce a signal that causes the microcontroller to operate the actuator according to the orientation of the vessel.

Example 49. The delivery device of example 43 wherein the at least one nebulizer delivery device removably dockable to the reservoir.

Example 50. A kit to suppress the exhalation of particles, the kit comprising:

a measured quantity of calcium chloride;

a container sized to receive a defined quantity of water to dissolve the calcium chloride therein; and instructions.

Example 51. The kit of Example 50 wherein the quantity of calcium chloride is hermetically packaged by itself.

Example 52. The kit of any of Examples 50 or 51, further comprising: a measured quantity of sodium chloride.

Example 53. The kit of Example 52 wherein the quantity of sodium chloride is hermetically packaged by itself, separate from the measured quantity of calcium chloride.

Example 54. The kit of any of Examples 50 to 53, further comprising:

a measured quantity of at least one of distilled or sterilized water hermetically packaged by itself, separate from the measured quantity of calcium chloride.

Example 55. The kit of any of Examples 50 to 54, further comprising:

an essential oil, fragrance oil or flavor extract selected from the group consisting of cacao oil, caramel oil, cinnamon bark oil, coffee oil, eucalyptus oil, palm oil, fig oil, grapefruit oil, hazelnut oil, honeydew melon oil, lavender or spike lavender oil, lemongrass oil, lime oil, black or green pepper oil, peppermint oil, rosemary oil, strawberry oil, smoke oil, tobacco vanilla oil, vanilla oil, chocolate extract, anise extract, rose, linalool, and combinations thereof.

Example 56. The kit of Example 52 wherein the calcium chloride and the sodium chloride are dry powders packaged separately from the quantity of water.

Example 57. The kit of Example 52 wherein the quantity of water is packaged separately from calcium chloride and the sodium chloride.

Example 58. The kit of Example 52 wherein the calcium chloride is packaged separately from the sodium chloride.

Example 59. A method of diagnosing subjects, the method comprising:

sampling exhaled breath for a subject;

determining a metric that characterizes an amount of exhaled virus shed in the exhaled breath; and

correlating the metric with a category of that indicates at least one of: a level of illness and/or a level of transmission or transmissibility or a level of suggested quarantine precautions to be taken.

Example 60. The method of claim Example 59 wherein sampling exhaled breath for a subject includes sampling exhaled breath for a subject over a defined number of respiration cycles.

Exampled 61. The method of claim Example 59 wherein sampling exhaled breath for a subject includes sampling exhaled breath for a subject over a defined period of time.

Example 62. The method of any of Examples 59 through 61 wherein determining a metric that characterizes an amount of exhaled virus shed in the exhaled breath includes determining an aerosol number that represents a virus load in the exhaled breath during at least one of defined number of respiration cycles or the defined period of time.

Example 63. The method of any of Examples 59 through 61 wherein determining a metric that characterizes an amount of exhaled virus shed in the exhaled breath includes determining a count or approximate count of exhaled virus in the exhaled breath.

Example 64. The method of any of Examples 59 through 61 wherein determining a metric that characterizes an amount of exhaled virus shed in the exhaled breath includes determining a percentage of exhaled virus in the exhaled breath

Example 65. The method of any of Examples 59 through 61 wherein determining a metric that characterizes an amount of exhaled virus shed in the exhaled breath includes determining a volume or weight of droplets in the exhaled breath.

Example 66. The method of Example 59 wherein correlating the metric with a category includes correlating the metric with respect to a representative sampling of breath samples taken from a representative sample of a population.

Example 67: An individual was administered Composition B, which is a 5% calcium chloride solution similar to Composition A, except that it does not contain any NaCl. The summary of the individual's exhaled aerosol particles per liter of air is set forth below:

Before 313+/−145

After 15+/−27

These results show that before administering Composition B, the individual exhaled 313+/−145 particles per liter and 30 minutes after the person exhaled 15+/−27 particles per liter, thus demonstrating the effectiveness of Composition B.

Applicants incorporate by reference the following: U.S. provisional patent application Ser. No. 62/687,970, filed Jun. 21, 2018; U.S. provisional patent application Ser. No. 62/652,069, filed Apr. 3, 2018; U.S. provisional patent application Ser. No. 62/628,395, filed Feb. 9, 2018; U.S. provisional patent application Ser. No. 62/556,974, filed Sep. 11, 2017; U.S. provisional patent application Ser. No. 62/727,123, filed Sep. 5, 2018; U.S. nonprovisional patent application Ser. No. 16/122,673, filed Sep. 5, 2018 (published as US2019-0105460); U.S. provisional patent application Ser. No. 63/048,421, filed Jul. 6, 2020; U.S. provisional patent application Ser. No. 63/121,448, filed Dec. 12, 2020; U.S. provisional patent application Ser. No. 63/130,099, filed Dec. 23, 2020; and International patent application Serial No. PCT/US2018/050250 (published as WO 2019/051403).

Adam P, Stiffman M, and Blake R L, Jr (1998) A clinical trial of hypertonic saline nasal spray in subjects with the common cold or rhinosinusitis. Arch Fam Med 7(1), 39-43; Alp S, et al (2005) Expression of beta-defensin 1 and 2 in nasal epithelial cells and alveolar macrophages from HIV-infected patients. Eur J Med Res 10(1), 1-6.; Bai, Y, et al (2020) Presumed Asymptomatic Carrier Transmission of COVID-19. JAMA; Bastier (2015). Nasal irrigation: From empiricism to evidence-based medicine. A review; Bunyan D, et al (2013) Respiratory and facial protection: a critical review of recent literature. J Hosp Infect. 85(3), 165-169; Calmet H, et al (2019) Nasal sprayed particle deposition in a human nasal cavity under different inhalation conditions. PLOS ONE 14(9), e0221330; Choy et al (2020) Remdesivir, lopinavir, emetine, and homoharringtonine inhibit SARS-CoV-2 T replication in vitro. Antiviral Research 178, 104786; Dellanno C, Vega Q, and Boesenberg D (2009) The antiviral action of common household disinfectants and antiseptics against murine hepatitis virus, a potential surrogate for SARS coronavirus. American Journal of Infection Control 37(8), 649-652; Edwards D A, et al (2004) Inhaling to mitigate exhaled bioaerosols. Proceedings of the National Academy of Sciences 101(50), 17383-17388; Fauci A S, Lane H C, and Redfield R R (2020) Covid-19—Navigating the Uncharted. New England Journal of Medicine 382 (13), 1268-1269; Fulcher M L, et al (2005) Well-Differentiated Human Airway Epithelial Cell Cultures, (ed. Picot J) Human Cell Culture Protocols. Methods in Molecular Medicine™, vol 107. Humana Press, 183-206. Geller C, Varbanov M, and Duval R (2012) Human Coronaviruses: Insights into Environmental Resistance and Its Influence on the Development of New Antiseptic Strategies. Viruses 4 (11), 3044-3068; Head, K, Snidvongs, K, Glew, S., Scadding, G., Schiller, A., Philpot, C and C Hopkins (2018). Saline irrigation for allergic rhinitis. Cochrane Database Syst Rev. CD01259; Hou, Y. J et al (2020) SARS-CoV-2 Reverse Genetics Reveals a Variable Infection Gradient in the Respiratory Tract. Cell 182, 1-18; Kim J, et al (2018) Human β-defensin 2 plays a regulatory role in innate antiviral immunity and is capable of potentiating the induction of antigen-specific immunity. Virology Journal 15(1); Krisanaprakornkit S, Jotikasthira D, and Dale B A (2003) Intracellular calcium in signaling human beta-defensin-2 expression in oral epithelial cells. J Dent Res 82(11), 877-82; Lescure, F-X, et al (2020) Clinical and virological data of the first cases of COVID-19 in Europe: a case series. The Lancet Infectious Diseases; Leung N H L, et al (2020) Respiratory virus shedding in exhaled breath and efficacy of face masks. Nature Medicine; Ngaosuwankul N, et al (2010) Influenza A viral loads in respiratory samples collected from patients infected with pandemic H1N1, seasonal H1N1 and H3N2 viruses. Virology Journal 7(1), 75. Ramalingam S, et al (2018) Antiviral innate immune response in non-myeloid cells is augmented by chloride ions via an increase in intracellular hypochlorous acid levels. Scientific Reports 8(1); Ramalingam S, et al (2019) A pilot, open labelled, randomised controlled trial of hypertonic saline nasal irrigation and gargling for the common cold. Scientific Reports 9(1); Schmittgen T D and Livak K J (2008) Analyzing real-time PCR data by the comparative C(T) method. Nat Protoc 3(6), 1101-8; Šlapak I, et al (2008) Efficacy of Isotonic Nasal Wash (Seawater) in the Treatment and Prevention of Rhinitis in Children. Arch Otolaryngol Head Neck Surg 134(1), 67; Speir R W (1961) Effect of Several Inorganic Salts on Infectivity of Mengo Virus. Proceedings of the Society for Experimental Biology and Medicine 106(2), 402-404; Sweet C and Smith H (1980) Pathogenicity of influenza virus. Microbiol Rev 44(2), 303-30; Van Doremalen N, et al (2020) Aerosol and Surface Stability of SARS-CoV-2 as Compared with SARS-CoV-1. New England Journal of Medicine 382, 1564-1567; Wang J, and Du G (2020) COVID-19 may transmit through aerosol. Irish Journal of Medical Science (1971-); Watanabe W, et al (2007) Why inhaling salt water changes what we exhale. J Colloid Interface Sci 307(1), 71-8; WHO Guidelines on Hand Hygiene in Health Care: First Global Patient Safety Challenge Clean Care Is Safer Care. Geneva: World Health Organization; 2009. 4, Historical perspective on hand hygiene in health care. Available from: https://www.ncbi.nlm.nih.gov/books/NBK144018/; Zhang, P. R. et al. (2020). Identifying airborne transmission as the dominant route for the spread of COVID-19. PNAS, 1-7; Zhao H, et al (2016) A novel peptide with potent and broad-spectrum antiviral activities against multiple respiratory viruses. Scientific Reports 6(1), 22008; Zou L, et al (2020) SARS-CoV-2 Viral Load in Upper Respiratory Specimens of Infected Patients. New England Journal of Medicine 382(12), 1177-1179.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1.-81. (canceled)

82. A composition of aerosol droplets comprising a salt-based composition, the droplets comprising:
(a) from about 1% to about 10% by weight calcium chloride and/or magnesium chloride in water; and
(b1) a preservative selected from the group consisting of benzalkonium chloride, benzoic acid, and benzoyl alcohol, or
(b2) an acid in an amount sufficient to reduce the pH of the salt-based composition to about 2 to about 6;
wherein the droplets have a mass median droplet diameter ranging from approximately 7 microns to approximately 15 microns.

83. The composition of claim 82, wherein the droplets have a mass median droplet diameter ranging from 8 microns to 12 microns, with a standard deviation of less than 5 microns.

84. The composition of claim 82, wherein the preservative is benzalkonium chloride, present in an amount ranging from 0.05-0.2 wt %.

85. The composition of claim 82, wherein the salt-based composition comprises about 4.0-6.0 wt % calcium chloride.

86. The composition of claim 82, wherein the salt-based composition further comprises an essential oil, fragrance oil or flavor extract selected from the group consisting of cacao oil, caramel oil, cinnamon bark oil, coffee oil, eucalyptus oil, palm oil, fig oil, grapefruit oil, hazelnut oil, honeydew melon oil, lavender or spike lavender oil, lemongrass oil, lime oil, black or green pepper oil, peppermint oil, rosemary oil, strawberry oil, smoke oil, tobacco vanilla oil, vanilla oil, chocolate extract, anise extract, rose oil, linalool extract, and combinations thereof.

87. A composition comprising:
(a) a dry powder containing calcium and/or magnesium chloride; and
(b) a sterile solution of a water-based composition comprising:
(1) a preservative selected from the group consisting of benzalkonium chloride, benzoic acid, and benzoyl alcohol, or
(2) an acid in an amount sufficient to reduce the pH of the salt-based composition to about 2 to about 6;
wherein the dry powder can be mixed with the water-based composition to form a salt-based composition.

88. The composition of claim 87, wherein the water-based composition comprises the acid (2).

89. The composition of claim 88, wherein the pH of the salt-based composition ranges from about 2 to about 4.

90. The composition of claim 87, wherein the acid is citric acid, hydrochloric acid, or a combination thereof.

91. The composition of claim 87, wherein the dry powder does not contain sodium chloride.

92. The composition of claim 87, wherein the dry powder is in a sachet.

93. A method of administering a formulation or composition to the nose, trachea, and main bronchi of a respiratory tract of a subject, method comprising:
generating an aerosol of droplets in a space from which the aerosol is naturally inspirable by the subject, in the nose, trachea, and main bronchi of the respiratory tract of the subject, without any application of force;
wherein the aerosol of droplets comprises a salt-based composition comprising calcium chloride and/or magnesium chloride in water, and
wherein the droplets have a mass median droplet diameter ranging from approximate 7 microns to approximately 15 microns.

94. The method of claim 93, wherein the salt-based composition does not contain sodium chloride.

95. The method of claim 93, wherein the droplets have a mass median droplet diameter ranging from 8 microns to 12 microns with a standard deviation of less than 5 microns.

96. The method of claim 93, wherein the subject inhales an amount of aerosol droplets comprising between 0.5-4.0 mg calcium chloride and/or magnesium chloride.

97. The method of claim 93, wherein the generating step comprises providing the aerosol into a free space.

98. The method of claim 93, wherein a velocity of the aerosol is slowed down relative to a velocity of the aerosol as it leaves a dispenser and from which the aerosol becomes relatively quiescent.

99. The method of claim 93, wherein the aerosol is provided in a range of 12 inches to 1 inch of a nose of the subject.

100. The method of claim 93, wherein the salt-based composition further comprises an essential oil, fragrance oil or flavor extract selected from the group consisting of cacao oil, caramel oil, cinnamon bark oil, coffee oil, eucalyptus oil, palm oil, fig oil, grapefruit oil, hazelnut oil, honeydew melon oil, lavender or spike lavender oil, lemongrass oil, lime oil, black or green pepper oil, peppermint oil, rosemary oil, strawberry oil, smoke oil, tobacco vanilla oil, vanilla oil, chocolate extract, anise extract, rose, linalool, and combinations thereof.

101. A delivery system operable to delivery of a purely hygienic or antimicrobial formulation or composition to the nose, trachea and main bronchi of a respiratory tract of a subject, the delivery system comprising:
a reservoir having at least one wall which at least partially delimits an interior of the reservoir from an exterior thereof, the reservoir having a port that provides a fluidly communicative path between the interior of the reservoir and an exterior thereof, the reservoir which at least in use holds the hygienic or antimicrobial formulation or composition comprising a quantity of water and at least calcium chloride and/or magnesium chloride dissolved in the water;
at least one nebulizer delivery device, the at least one nebulizer delivery device comprising a reservoir and an actuator, and the actuator controllably operable on the active substance media to cause formation of an aerosol comprising readily-soluble droplets that have a mass median diameter range of approximately 7 microns to approximately 15 microns and comprising at least the calcium chloride dissolved in the quantity of water.

102. The delivery system of claim 101, wherein the at least one nebulizer delivery device is a nebulizer that includes a mesh screen mounted for oscillation, a microcontroller, and at least one of a piezoelectric transducer, a solenoid, or an electric motor drivingly coupled to oscillate the mesh screen along at least one axis in response to signals from the microcontroller to dispense aerosol.

103. The delivery system of claim 101, wherein the at least one nebulizer delivery device removably dockable to the reservoir.

104. A kit to suppress the exhalation of particles in an upper airway of a respiratory tract of subjects, the kit including the composition according to claim 87, wherein the dry powder comprises a measured quantity of calcium chloride and/or magnesium chloride, and further comprising:
a container sized to receive a defined quantity of the sterile solution to dissolve the dry powder therein; and
instructions.

105. The kit of claim 104, wherein the measured quantity of calcium chloride and/or magnesium chloride is hermetically packaged by itself.

106. The kit of claim 104 wherein the defined quantity of the sterile solution is hermetically packaged by itself, separate from the measured quantity of calcium chloride.

107. The composition of claim 82, wherein composition provides at least a 45% reduction in exhaled particles in a group of human subjects of at least ten individuals approximately one hour after nasal inhalation relative to the number of exhaled particles by the group prior to nasal inhalation of the composition and as measured by a particle counter that samples the exhaled particles of the subjects while filtering the air via a HEPA filter of environmental particles so that the exhaled particle numbers are representative of the particles generated within the individual's airways.

108. The composition of claim 82, wherein composition provides at least a 45% reduction in exhaled particles in a group of human subjects of at least ten individuals approximately one hour after nasal inhalation relative to the number of exhaled particles by the group prior to nasal inhalation of the composition and as measured by a Climet 450-t particle counter that samples the exhaled particles of the subjects while filtering the air via a HEPA filter of environmental particles so that the exhaled particle numbers are representative of the particles generated within the individual's airways.

109. The composition of claim 82, wherein composition provides at least a 75% reduction in exhaled particles in a group of human subjects of at least ten individuals approximately one hour after nasal inhalation relative to the number of exhaled particles by the group prior to nasal inhalation of the composition and as measured by a particle counter that samples the exhaled particles of the subjects while filtering the air via a HEPA filter of environmental particles so that the exhaled particle numbers are representative of the particles generated within the individual's airways.

110. The composition of claim 82, wherein composition provides at least a 75% reduction in exhaled particles in a group of human subjects of at least ten individuals approximately one hour after nasal inhalation relative to the number of exhaled particles by the group prior to nasal inhalation of the composition and as measured by a Climet 450-t particle counter that samples the exhaled particles of the subjects while filtering the air via a HEPA filter of environmental particles so that the exhaled particle numbers are representative of the particles generated within the individual's airways.

111. The composition of claim 82, wherein composition provides at least an 86% reduction in exhaled particles in a group of human subjects of at least ten individuals approximately two hours after nasal inhalation relative to the number of exhaled particles by the group prior to nasal inhalation of the composition and as measured by a particle counter that samples the exhaled particles of the subjects while filtering the air via a HEPA filter of environmental particles so that the exhaled particle numbers are representative of the particles generated within the individual's airways.

112. The composition of claim 82, wherein composition provides at least an 86% reduction in exhaled particles in a group of human subjects of at least ten individuals approximately two hours after nasal inhalation relative to the number of exhaled particles by the group prior to nasal inhalation of the composition and as measured by a Climet 450-t particle counter that samples the exhaled particles of the subjects while filtering the air via a HEPA filter of environmental particles so that the exhaled particle numbers are representative of the particles generated within the individual's airways.

113. The method of claim 93, wherein natural inspiration of the composition provides at least a 45% reduction in exhaled particles in a group of human subjects of at least ten individuals approximately one hour after nasal inhalation relative to the number of exhaled particles by the group prior to nasal inhalation of the composition and as measured by a particle counter that samples the exhaled particles of the subjects while filtering the air via a HEPA filter of environmental particles so that the exhaled particle numbers are representative of the particles generated within the individual's airways.

114. The method of claim 93, wherein natural inspiration of the composition provides at least a 45% reduction in exhaled particles in a group of human subjects of at least ten individuals approximately one hour after nasal inhalation relative to the number of exhaled particles by the group prior to nasal inhalation of the composition and as measured by a Climet 450-t particle counter that samples the exhaled particles of the subjects while filtering the air via a HEPA filter of environmental particles so that the exhaled particle numbers are representative of the particles generated within the individual's airways.

115. The method of claim 93, wherein natural inspiration of composition provides at least a 75% reduction in exhaled particles in a group of human subjects of at least ten individuals approximately one hour after nasal inhalation relative to the number of exhaled particles by the group prior to nasal inhalation of the composition and as measured by a particle counter that samples the exhaled particles of the subjects while filtering the air via a HEPA filter of environmental particles so that the exhaled particle numbers are representative of the particles generated within the individual's airways.

116. The method of claim 93, wherein natural inspiration of composition provides at least a 75% reduction in exhaled particles in a group of human subjects of at least ten individuals approximately one hour after nasal inhalation relative to the number of exhaled particles by the group prior to nasal inhalation of the composition and as measured by a Climet 450-t particle counter that samples the exhaled particles of the subjects while filtering the air via a HEPA filter of environmental particles so that the exhaled particle numbers are representative of the particles generated within the individual's airways.

117. The method of claim 93, wherein natural inspiration of composition provides at least an 86% reduction in exhaled particles in a group of human subjects of at least ten individuals approximately two hours after nasal inhalation relative to the number of exhaled particles by the group prior to nasal inhalation of the composition and as measured by a particle counter that samples the exhaled particles of the subjects while filtering the air via a HEPA filter of environmental particles so that the exhaled particle numbers are representative of the particles generated within the individual's airways.

118. The method of claim 93, wherein natural inspiration of composition provides at least an 86% reduction in exhaled particles in a group of human subjects of at least ten individuals approximately two hours after nasal inhalation relative to the number of exhaled particles by the group prior to nasal inhalation of the composition and as measured by a Climet 450-t particle counter that samples the exhaled particles of the subjects while filtering the air via a HEPA filter of environmental particles so that the exhaled particle numbers are representative of the particles generated within the individual's airways.
